SEQUENCE LISTING (6 sequences)

SEQ ID NO: 1 - DNA, 3286 bp, Homo sapiens
  CDS (1116)..(2417)
gcgtgtcgggcgcggaagggggaggcggcccggggcgcccgcgagtgaggcgcggggcggcgaagggagcgcgggtggcggcacttgctgccgcggccttggatgggctgggcccccctcgccgctccgcctcctccacacgcgcggcggccgcggcgagggggacgcgccgcccggggcccggcaccttcgggaaccccccggcccggagcctgcggcctgcgccgcctcggccgccgggagccccgtggagcccccgccgccgcgccgccccgcggaccggacgctgagggcactcggggcggggcgcgcgctcgggcagacgtttgcggggaggggggcgcctgccgggccccggcgaccaccttgggggtcgcgggccggctcggggggcgcccagtgcgggccctcgcgggcgccgggcagcgaccagccctgagcggagctgttggccgcggcgggaggcctcccggacgcccccagccccccgaacgctcgcccgggccggcgggagtcggcgccccccgggaggtccgctcggtcgtccgcggcggagcgtttgctcctgggacaggcggtgggaccggggcgtcgccggagacgcccccagcgaagttgggctctccaggtgtgggggtcccggggggtagcgacgtcgcggacccggcctgtgggatgggcggcccggagaagactgcgctcggccgtgttcatacttgtccgtgggcctgaggtccccggaggatgacctagcactgaaaagccccggccggcctccccagggtccccgaggacgaagttgaccctgaccgggccgtctcccagttctgaggcccgggtcccactggaactcgcgtctgagccgccgtcccggacccccggtgcccgccggtccgcagaccctgcaccgggcttggactcgcagccgggactgacgtgtagaacaatcgtttctgttggaagaagggtttttcccttccttttggggtttttgttgccttttttttttcttttttctttgtaaaattttggagaagggaagtcggaacacaaggaaggaccgctcacccgcggactcagggctggcggcgggactccaggaccctgggtccagcatggaggtggtggacccgcagcagctgggcatgttcacggagggcgagctgatgtcggtgggtatggacacgttcatccaccgcatcgactccaccgaggtcatctaccagccgcgccgcaagcgggccaagctcatcggcaagtacctgatgggggacctgctgggggaaggctcttacggcaaggtgaaggaggtgctggactcggagacgctgtgcaggagggccgtcaagatcctcaagaagaagaagttgcgaaggatccccaacggggaggccaacgtgaagaaggaaattcaactactgaggaggttacggcacaaaaatgtcatccagctggtggatgtgttatacaacgaagagaagcagaaaatgtatatggtgatggagtactgcgtgtgtggcatgcaggaaatgctggacagcgtgccggagaagcgtttcccagtgtgccaggcccacgggtacttctgtcagctgattgacggcctggagtacctgcatagccagggcattgtgcacaaggacatcaagccggggaacctgctgctcaccaccggtggcaccctcaaaatctccgacctgggcgtggccgaggcactgcacccgttcgcggcggacgacacctgccggaccagccagggctccccggctttccagccgcccgagattgccaacggcctggacaccttctccggcttcaaggtggacatctggtcggctggggtcaccctctacaacatcaccacgggtctgtaccccttcgaaggggacaacatctacaagttgtttgagaacatcgggaaggggagctacgccatcccgggcgactgtggccccccgctctctgacctgctgaaagggatgcttgagtacgaaccggccaagaggttctccatccggcagatccggcagcacagctggttccggaagaaacatcctccggctgaagcaccagtgcccatcccaccgagcccagacaccaaggaccggtggcgcagcatgactgtggtgccgtacttggaggacctgcacggcgcggacgaggacgaggacctcttcgacatcgaggatgacatcatctacactcaggacttcacggtgcccggacaggtcccagaagaggaggccagtcacaatggacagcgccggggcctccccaaggccgtgtgtatgaacggcacagaggcggcgcagctgagcaccaaatccagggcggagggccgggcccccaaccctgcccgcaaggcctgctccgccagcagcaagatccgccggctgtcggcctgcaagcagcagtgaggctggccgcctgcagcccgtgtccaggagccccgccaggtgcccgcgccaggccctcagtcttcctgccggttccgcccgccctcccggagaggtggccgccatgcttctgtgccgaccacgccccaggacctccggagcgccctgcagggccgggcagggggacagcagggaccgggcgcagccctcccccctcggccgcccggcagtgcacgcggcttgttgacttcgcagccccgggcggagccttcccgggcgggcgtgggaggagggaggcggcctccatgcactttatgtggagactactggccccgcccgtggcctcgtgctccgcagggcgcccagcgccgtccggcggccccgccgcagaccagctggcgggtgtggagaccaggctcctgaccccgccatgcatgcagcgccacctggaagccgcgcggccgctttggttttttgtttggttggttccattttctttttttctttttttttttaagaaaaaataaaaggtggatttgagctgtggctgtgaggggtgtttgggagctgctgggtggcaggggggctgtggggtcgggctcacgtcgcggccgcctttgcgctctcgggtcaccctgctttggcggcccggccggagggcaggaccctcacctctcccccaaggccactgcgctcttgggaccccagagaaaacccggagcaagcaggagtgtgcggtcaatatttatatcatccagaaaagaaaaacacgagaaacgccatcgcgggatggtgcagacgcggcggggactcggagggtgccgtgcgggcgaggccgcccaaatttggcaataaataaagcttgggaagcttggacctgaaaaaaaaaa

SEQ ID NO: 2 - protein, 433 aa, Homo sapiens
MEVVDPQQLGMFTEGELMSVGMDTFIHRIDSTEVIYQPRRKRAKLIGKYLMGDLLGEGSYGKVKEVLDSETLCRRAVKILKKKKLRRIPNGEANVKKEIQLLRRLRHKNVIQLVDVLYNEEKQKMYMVMEYCVCGMQEMLDSVPEKRFPVCQAHGYFCQLIDGLEYLHSQGIVHKDIKPGNLLLTTGGTLKISDLGVAEALHPFAADDTCRTSQGSPAFQPPEIANGLDTFSGFKVDIWSAGVTLYNITTGLYPFEGDNIYKLFENIGKGSYAIPGDCGPPLSDLLKGMLEYEPAKRFSIRQIRQHSWFRKKHPPAEAPVPIPPSPDTKDRWRSMTVVPYLEDLHGADEDEDLFDIEDDIIYTQDFTVPGQVPEEEASHNGQRRGLPKAVCMNGTEAAQLSTKSRAEGRAPNPARKACSASSKIRRLSACKQQ

SEQ ID NO: 3 - DNA, 4685 bp, Homo sapiens
  CDS (222)..(1853)
ggaggaggtggagagtgaggccgaggcgtggggagcccgggaactccctcctcctgaagtaacgcgtcccgggccggctctgccgtcgttgctgccgccgggcgccccgggacgaggaggtggaggagggagagggcccgcgggcctcgcctccgccctccgccacctcgagctgcggtagcagcgactcatgagagcgcggccggaggacagatttgataatgggctgcattaaaagtaaagaaaacaaaagtccagccattaaatacagacctgaaaatactccagagcctgtcagtacaagtgtgagccattatggagcagaacccactacagtgtcaccatgtccgtcatcttcagcaaagggaacagcagttaatttcagcagtctttccatgacaccatttggaggatcctcaggggtaacgccttttggaggtgcatcttcctcattttcagtggtgccaagttcatatcctgctggtttaacaggtggtgttactatatttgtggccttatatgattatgaagctagaactacagaagacctttcatttaagaagggtgaaagatttcaaataattaacaatacggaaggagattggtgggaagcaagatcaatcgctacaggaaagaatggttatatcccgagcaattatgtagcgcctgcagattccattcaggcagaagaatggtattttggcaaaatggggagaaaagatgctgaaagattacttttgaatcctggaaatcaacgaggtattttcttagtaagagagagtgaaacaactaaaggtgcttattccctttctattcgtgattgggatgagataaggggtgacaatgtgaaacactacaaaattaggaaacttgacaatggtggatactatatcacaaccagagcacaatttgatactctgcagaaattggtgaaacactacacagaacatgctgatggtttatgccacaagttgacaactgtgtgtccaactgtgaaacctcagactcaaggtctagcaaaagatgcttgggaaatccctcgagaatctttgcgactagaggttaaactaggacaaggatgtttcggcgaagtgtggatgggaacatggaatggaaccacgaaagtagcaatcaaaacactaaaaccaggtacaatgatgccagaagctttccttcaagaagctcagataatgaaaaaattaagacatgataaacttgttccactatatgctgttgtttctgaagaaccaatttacattgtcactgaatttatgtcaaaaggaagcttattagatttccttaaggaaggagatggaaagtatttgaagcttccacagctggttgatatggctgctcagattgctgatggtatggcatatattgaaagaatgaactatattcaccgagatcttcgggctgctaatattcttgtaggagaaaatcttgtgtgcaaaatagcagactttggtttagcaaggttaattgaagacaatgaatacacagcaagacaaggtgcaaaatttccaatcaaatggacagctcctgaagctgcactgtatggtcggtttacaataaagtctgatgtctggtcatttggaattctgcaaacagaactagtaacaaagggccgagtgccatatccaggtatggtgaaccgtgaagtactagaacaagtggagcgaggatacaggatgccgtgccctcagggctgtccagaatccctccatgaattgatgaatctgtgttggaagaaggaccctgatgaaagaccaacatttgaatatattcagtccttcttggaagactacttcactgctacagagccacagtaccagccaggagaaaatttataattcaagtagcctattttatatgcacaaatctgccaaaatataaagaacttgtgtagattttctacaggaatcaaaagaagaaaatcttctttactctgcatgtttttaatggtaaactggaatcccagatatggttgcacaaaaccacttttttttccccaagtattaaactctaatgtaccaatgatgaatttatcagcgtatttcagggtccaaacaaaatagagctaagatactgatgacagtgtgggtgacagcatggtaatgaaggacagtgaggctcctgcttatttataaatcatttcctttctttttttccccaaagtcagaattgctcaaagaaaattatttattgttacagataaaacttgagagataaaaagctataccataataaaatctaaaattaaggaatatcatgggaccaaataattccattccagttttttaaagtttcttgcatttattattctcaaaagttttttctaagttaaacagtcagtatgcaatcttaatatatgctttcttttgcatggacatgggccaggtttttcaaaaggaatataaacaggatctcaaacttgattaaatgttagaccacagaagtggaatttgaaagtataatgcagtacattaatattcatgttcatggaactgaaagaataagaactttttcacttcagtccttttctgaagagtttgacttagaataatgaaggtaactagaaagtgagttaatcttgtatgaggttgcattgattttttaaggcaatatataattgaaactactgtccaatcaaaggggaaatgttttgatctttagatagcatgcaaagtaagacccagcattttaaaagccctttttaaaaactagacttcgtactgtgagtattgcttatatgtccttatggggatgggtgccacaaatagaaaatatgaccagatcagggacttgaatgcacttttgctcatggtgaatatagatgaacagagaggaaaatgtatttaaaagaaatacgagaaaagaaagtgaaagttttacaagttagagggatggaaggtaatgtttaatgttgatgtcatggagtgacagaatggctttgctggcactcagagctcctcacttagctatattctgagactttgaagagttataaagtataactataaaactaatttttcttacacactaaatgggtatttgttcaaaataatgaagttatggcttcacattcattgcagtgggatatggtttttatgtaaaacatttttagaactccagttttcaaatcatgtttgaatctacattcacttttttttgttttcttttttgagacggagtctcgctctgtcgcccaggctggagtgcagtggcgcgatctcggctcactgcaagctctgcctcccaggttcacaccattctcctgcctcagcctcccgagtagctgggactacaggtgcccaccaccacgcctggctagttttttgtatttttagtagagacgcagtttcaccgtgttagccaggatggtctcgatctcctgaccttgtgatctgcccgcctcggcctcccaaagtgctgggattacaggcgtgagccaccgcgcccagcctacattcacttctaaagtctatgtaatggtggtcattttttcccttttagaatacattaaatggttgatttggggaggaaaacttattctgaatattaacggtggtgaaaaggggacagtttttaccctaaagtgcaaaagtgaaacatacaaaataagactaatttttaagagtaactcagtaatttcaaaatacagatttgaatagcagcattagtggtttgagtgtctagcaaaggaaaaattgatgaataaaatgaaggtctggtgtatatgttttaaaatactctcatatagtcacactttaaattaagccttatattaggcccctctattttcaggatataattcttaactatcattatttacctgattttaatcatcagattcgaaattctgtgccatggcatatatgttcaaattcaaaccatttttaaaatgtgaagatggacttcatgcaagttggcagtggttctggtactaaaaattgtggttgttttttctgtttacgtaacctgcttagtattgacactctctaccaagagggtcttcctaagaagagtgctgtcattatttcctcttatcaacaacttgtgacatgagattttttaagggctttatgtgaactatgatattgtaatttttctaagcatattcaaaagggtgacaaaattacgtttatgtactaaatctaatcaggaaagtaaggcaggaaaagttgatggtattcattaggttttaactgaatggagcagttccttatataataacaattgtatagtagggataaaacactaacttaatgtgtattcattttaaattgttctgtatttttaaattgccaagaaaaacaactttgtaaatttggagatattttccaacagcttttcgtcttcagtgtcttaatgtggaagttaacccttaccaaaaaaggaagttggcaaaaacagccttctagcacacttttttaaatgaataatggtagcctaaacttaatatttttataaagtattgtaatattgttttgtggataattgaaataaaaagttctcattgaatgcacctattaatcgttttagttgctattcatattctcattcgttttttaaaaactgatatattctgaatttattcttccattgagaaaaaaatgttcagttacttgtaactactgagcagaatttaatcaatcctttattaaattcagaacattattgaa

SEQ ID NO: 4 - protein, 543 aa, Homo sapiens
MGCIKSKENKSPAIKYRPENTPEPVSTSVSHYGAEPTTVSPCPSSSAKGTAVNFSSLSMTPFGGSSGVTPFGGASSSFSVVPSSYPAGLTGGVTIFVALYDYEARTTEDLSFKKGERFQIINNTEGDWWEARSIATGKNGYIPSNYVAPADSIQAEEWYFGKMGRKDAERLLLNPGNQRGIFLVRESETTKGAYSLSIRDWDEIRGDNVKHYKIRKLDNGGYYITTRAQFDTLQKLVKHYTEHADGLCHKLTTVCPTVKPQTQGLAKDAWEIPRESLRLEVKLGQGCFGEVWMGTWNGTTKVAIKTLKPGTMMPEAFLQEAQIMKKLRHDKLVPLYAVVSEEPIYIVTEFMSKGSLLDFLKEGDGKYLKLPQLVDMAAQIADGMAYIERMNYIHRDLRAANILVGENLVCKIADFGLARLIEDNEYTARQGAKFPIKWTAPEAALYGRFTIKSDVWSFGILQTELVTKGRVPYPGMVNREVLEQVERGYRMPCPQGCPESLHELMNLCWKKDPDERPTFEYIQSFLEDYFTATEPQYQPGENL

SEQ ID NO: 5 - DNA, 2194 bp, Homo sapiens
  CDS (111)..(353)
gggtctcgccggctcgccgcgctccccaccttgcctgcgcccgcccggagccagcggttctccaagcacccagcatcctgctagacgcgccgcgcaccgacggaggggacatgggcagagcaatggtggccaggctcgggctggggctgctgctgctggcactgctcctacccacgcagatttattccagtgaaacaacaactggaacttcaagtaactcctcccagagtacttccaactctgggttggccccaaatccaactaatgccaccaccaaggcggctggtggtgccctgcagtcaacagccagtctcttcgtggtctcactctctcttctgcatctctactcttaagagactcaggccaagaaacgtcttctaaatttccccatcttctaaacccaatccaaatggcgtctggaagtccaatgtggcaaggaaaaacaggtcttcatcgaatctactaattccacaccttttattgacacagaaaatgttgagaatcccaaatttgattgatttgaagaacatgtgagaggtttgactagatgatggatgccaatattaaatctgctggagtttcatgtacaagatgaaggagaggcaacatccaaaatagttaagacatgatttccttgaatgtggcttgagaaatatggacacttaatactaccttgaaaataagaatagaaataaaggatgggattgtggaatggagattcagttttcatttggttcattaattctataaggccataaaacaggtaatataaaaagcttccatgattctatttatatgtacatgagaaggaacttccaggtgttactgtaattcctcaacgtattgtttcgacagcactaatttaatgccgatatactctagatgaagttttacattgttgagctattgctgttctcttgggaactgaactcactttcctcctgaggctttggatttgacattgcatttgaccttttatgtagtaattgacatgtgccagggcaatgatgaatgagaatctacccccagatccaagcatcctgagcaactcttgattatccatattgagtcaaatggtaggcatttcctatcacctgtttccattcaacaagagcactacattcatttagctaaacggattccaaagagtagaattgcattgaccacgactaatttcaaaatgctttttattattattattttttagacagtctcactttgtcgcccaggccggagtgcagtggtgcgatctcagatcagtgtaccatttgcctcccgggctcaagcgattctcctgcctcagcctcccaagtagctgggattacaggcacctgccaccatgcccggctaatttttgtaattttagtagagacagggtttcaccatgttgcccaggctggtttcgaactcctgacctcaggtgatccacccgcctcggcctcccaaagtgctgggattacaggcttgagcccccgcgcccagccatcaaaatgctttttatttctgcatatgttgaatactttttacaatttaaaaaaatgatctgttttgaaggcaaaattgcaaatcttgaaattaagaaggcaaaaatgtaaaggagtcaaaactataaatcaagtatttgggaagtgaagactggaagctaatttgcattaaattcacaaacttttatactctttctgtatatacattttttttctttaaaaaacaactatggatcagaatagccacatttagaacactttttgttatcagtcaatatttttagatagttagaacctggtcctaagcctaaaagtgggcttgattctgcagtaaatcttttacaactgcctcgacacacataaacctttttaaaaatagacactccccgaagtcttttgttcgcatggtcacacactgatgcttagatgttccagtaatctaatatggccacagtagtcttgatgaccaaagtcctttttttccatctttagaaaactacatgggaacaaacagatcgaacagttttgaagctactgtgtgtgtgaatgaacactcttgctttattccagaatgctgtacatctattttggattgtatattgtgtttgtgtatttacgctttgattcatagtaacttcttatggaattgatttgcattgaacacaaactgtaaataaaaagaaatggctgaaagagcaaaaaaaaaaaaa

SEQ ID NO: 6 - protein, 80 aa, Homo sapiens
MGRAMVARLGLGLLLLALLLPTQIYSSETTTGTSSNSSQSTSNSGLAPNPTNATTKAAGGALQSTASLFVVSLSLLHLYS

(19) United States
(12) Patent Application Publication
(10) Pub. No.: US 2014/0378469 A1
(43) Pub. Date: Dec. 25, 2014

(54) LKB1/STK11 DELETION IN MELANOMA AND RELATED METHODS

(71) Applicants: The University of North Carolina at Chapel Hill, Chapel Hill, NC (US); Dana-Farber Cancer Institute, Inc., Boston, MA (US)

(72) Inventors: Norman Edward Sharpless, Chapel Hill, NC (US); Wenjin Liu, Chapel Hill, NC (US); James Bear, Chapel Hill, NC (US); Kimberly Monahan, Chapel Hill, NC (US); Kwok-Kin Wong, Arlington, MA (US)

(21) Appl. No.: 14/376,997

(22) PCT Filed: Feb. 6, 2013
(86) PCT No.: PCT/US2013/024947

§ 371 Date: Aug. 6, 2014

Related U.S. Application Data
(60) Provisional application No. 61/595,512, filed on Feb. 6, 2012.

(51) Int. Cl. C12Q 1/68; G01N 33/574

(52) U.S. Cl. CPC C12Q 1/6886; G01N 33/5743; G01N 2333/912; G01N 2333/70596; C12Q 2600/158; C12Q 2600/156; C12Q 2600/118; C12Q 2600/106; G01N 2800/52
USPC 514/252.17; 435/6.11; 435/6.14; 435/6.12; 506/9; 506/16; 435/7.4; 435/7.23

(57) ABSTRACT

LKB1 mutation status and/or expression, YES expression and phosphorylation level; and/or CD24 expression are employed to predict melanoma prognosis and response to therapeutics. Inhibitors (including targeted inhibitors) of SRC family kinases (especially YES) are employed to treat melanoma.

### RELATED APPLICATIONS

The presently disclosed subject matter is based on and claims the benefit of U.S. Provisional Patent Application Ser. No. 61/595,512, filed Feb. 6, 2012; the disclosure of which is incorporated herein by reference in its entirety.

### GOVERNMENT INTEREST

This presently disclosed subject matter was made with government support under Grant No. 5P01ES014635-05 awarded by National Institute of Environmental Health Sciences, National Institutes of Health of the United States. The government has certain rights in the presently disclosed subject matter.

### TECHNICAL FIELD

The presently disclosed subject matter relates to a LKB1/STK11 deletion in melanoma. In some embodiments, the presently disclosed subject matter relates to predicting outcomes for subjects having melanoma.

### BACKGROUND

Metastatic melanoma has a poor prognosis. Predicting prognosis in the disease plays a role in determining patient therapy. Advanced melanoma is treatment refractory, and new therapeutic approaches are needed for patients with advanced melanoma. As such, defining additional prognostic approaches would be beneficial by preventing patients from unnecessarily undergoing therapies, by allowing future therapies to be appropriately tailored, and by providing insight into the biology that underlies the disease of melanoma.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

Disclosed herein is a method of predicting a melanoma prognosis, the method comprising:

(a) detecting one or more of the following in a biological sample comprising melanoma cells obtained from a melanoma of a subject:
(i) the presence or absence of a LKB1 mutation, or a LKB1 expression level;
(ii) a YES expression level, a YES phosphorylation level, or both; and
(iii) a CD24 expression level; and

(b) predicting a melanoma prognosis based on the detecting of step (a).

Also disclosed herein is a method of predicting a response to a therapy by a melanoma in a subject having the melanoma and receiving the therapy, the method comprising:

(a) detecting one or more of the following in a biological sample comprising melanoma cells obtained from a melanoma of a subject:
(i) the presence or absence of a LKB1 mutation, or a LKB1 expression level;
(ii) a YES expression level, a YES phosphorylation level, or both; and
(iii) a CD24 expression level; and

(b) predicting a response to the therapeutic based on the detecting of step (a).

Also disclosed herein is method for managing treatment of a subject with melanoma, the method comprising:

(a) detecting one or more of the following in a biological sample comprising melanoma cells obtained from a melanoma of a subject:
(i) the presence or absence of a LKB1 mutation, or a LKB1 expression level;
(ii) a YES expression level, a YES phosphorylation level, or both; and
(iii) a CD24 expression level; and

(b) managing treatment of the subject based on the detecting of step (a).

Also disclosed herein is a method of selecting a therapy for a melanoma in a subject, comprising providing a subject suffering from a melanoma wherein LKB1, YES and/or CD24 status for the subject's melanoma has been assessed; and selecting a therapy to treat the melanoma in the subject based on the LKB1, YES and/or CD24 status.

Also disclosed herein is a method of treating melanoma in a subject in need thereof, comprising providing a subject suffering from a melanoma wherein LKB1, YES and/or CD24 status for the subject's melanoma has been assessed; and administering to the subject an effective amount of a therapeutic agent to treat the melanoma in the subject based on the LKB1, YES and/or CD24 status.

In some embodiments, the presence of an LKB1 mutation or of a reduced level of expression of LKB1 is indicative of a negative prognosis, a resistance to the therapy, or suggests an altered (e.g. more aggressive) treatment choice. In some embodiments, the LKB1 mutation results in decreased LKB1 expression, activity, or both expression and activity. In some embodiments, the absence of an LKB1 mutation or of a reduced level of expression of LKB1 is indicative of a positive prognosis, a lack of resistance to the therapy, or a conservative treatment choice.

In some embodiments, an elevated level of YES expression, YES phosphorylation, or both, is indicative of a negative prognosis, a resistance to the therapy, or suggests an altered (e.g. more aggressive) treatment choice. In some embodiments, the absence of an elevated level of YES expression, YES phosphorylation, or both, is indicative of a positive prognosis, a lack of resistance to the therapy, or a conservative treatment choice.

In some embodiments, an elevated level of CD24 expression is indicative of a negative prognosis, a resistance to the therapy, or suggests an altered (e.g. more aggressive) treatment choice. In some embodiments, the absence of an elevated level of CD24 expression is indicative of a positive prognosis, a lack of resistance to the therapy, or a conservative treatment choice.

In some embodiments, a risk of an adverse outcome of a subject with melanoma is assessed. In some embodiments, a clinical outcome of a treatment in a subject diagnosed with melanoma is predicted.

In some embodiments, an expression level is determined by a PCR-based method, a microarray based method, or an antibody-based method. In some embodiments, an expression level is normalized relative to an expression level of one or more reference genes. In some embodiments, the expression level is compared to a standard.

In some embodiments the therapy or treatment is selected from the group consisting of surgical resection of the melanoma, chemotherapy, molecular targeted therapy, immunotherapy, and combinations thereof.

Also disclosed herein is a method of treating melanoma in a subject in need thereof, comprising administering to the subject an effective amount of an inhibitor of a SRC family kinase, optionally a targeted inhibitor of a SRC family kinase, optionally YES, to treat a melanoma in the subject. The subject can be a mammal.

Also disclosed herein is a kit comprising one or more binding molecules for a gene selected from the group consisting of LKB1, YES, and CD24 and/or for a peptide or polypeptide gene product of LKB1, YES, or CD24.

Also disclosed herein is array comprising polynucleotides hybridizing to at least two genes selected from the group consisting of LKB1, YES, and CD24 or comprising specific peptide or polypeptide gene products of at least two of LKB1, YES, and CD24.

It is an object of the presently disclosed subject matter to provide methods for predicting outcome of subjects with melanoma.

An object of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE DRAWINGS

FIG. 1 is a graph showing the growth curves of primary melanocyte cultures from neonatal mice of various genotypes, including those that resulted from intercrossing an established 4-hydroxytamoxifen (4-OHT)-inducible melanocyte specific CRE allele (i.e., Tyr-Cre-ER^{T2} (T)) and three conditional alleles: Lox-Stop-Lox-(LSL)-Kras^{G12D} (K); Lkb1^{L/L}, and p53^{L/L}. Data is shown as follow: wild-type (WT), diamonds; TK, squares; TLKB1^{L/L}, triangles; and TKLKB1^{L/L}, X's. Cells were treated with 4-OHT at 20 days post isolation to activate CRE recombinase. Cell numbers were counted during serial passage. At least three primary lines were generated from each group and representative results were shown.

FIG. 2 is a graph showing Kaplan-Meier analysis of melanoma-free survival of cohorts of mice of various genotypes (i.e., TK, TLkb1^{L/L}, TKp53^{L/L}, TKLkb1^{L/L}, and TKp53^{L/L}Lkb1^{L/L}).

FIG. 3A is a bar graph of the mean close index of Lkb1-deficient (TKLkb1^{L/L} and TKp53^{L/L}Lkb1^{L/L}) and Lkb1-competent (TKp53^{L/L}p16^{L/L} and TRIA) melanoma cells subjected to in vitro wound healing or scratch assay. The mean close indexes were determined from three replicates per genotype.

FIG. 3B is a bar graph showing tumor invasiveness (quantified as mean number of invaded cells) determined by matrigel invasion assay of Lkb1-deficient (TKLkb1^{L/L} and TKp53^{L/L}Lkb1^{L/L}) and Lkb1-competent (TKp53^{L/L}p16^{L/L} and TRIA) melanoma cells. The means were determined from three replicates per genotype.

FIG. 3C is a bar graph showing the mean close index of isogenic cells with and without Lkb1 subjected to in vitro scratch assay. Lkb1 expression was restored in Lkb1-null melanoma cells (TKp53^{L/L}Lkb1^{L/L}) by transduction with wild-type Lkb1 or kinase-dead Lkb1 (Lkb1-KD). For comparison, close index was also measured in Lkb1-null cells that had been transduced with a control vector (Vector) and that still showed no Lkb1 expression. Lkb1-expression in Lkb1-competent cells (TKp53^{L/L}p16^{L/L}) was knocked down by transduction with a short hairpin RNA (shRNA) targeting Lkb1. For comparison, close index was also measured in Lkb1-competent cells that had been transduced with a non-specific shRNA (NS) and that still expressed Lkb1. The asterisks indicate a significant difference (P<0.05).

FIG. 3D is a bar graph showing tumor invasiveness (quantified as the mean number of invaded cells) in isogenic cells with and without Lkb1 subjected to matrigel invasion assay. Lkb1 expression was restored in Lkb1-null melanoma cells (TKp53^{L/L}Lkb1^{L/L}) by transduction with wild-type Lkb1 or kinase-dead Lkb1 (Lkb1-KD). For comparison, close index was also measured in Lkb1-null cells that had been transduced with a control vector (Vector) and that still showed no Lkb1 expression. Lkb1-expression in Lkb1-competent cells (TKp53^{L/L}p16^{L/L}) was knocked down by transduction with a short hairpin RNA (shRNA) targeting Lkb1. For comparison, close index was also measured in Lkb1-competent cells that had been transduced with a non-specific shRNA (NS) and that still expressed Lkb1.

FIG. 4A is a series of photographs of representative Western blot analyses of TKp53^{L/L}p16^{L/L} cells with (shLkb1) or without (NS) Lkb1 knockdown. Cell lysates were either directly immunoblotted (IB) with antibody (Y416) against pan-SRC family kinases (P-SFK) or immunoprecipitated (IP) first with the indicated antibodies against Src, Fyn, or Yes and then immunoblotted with antibody against P-SFK.

FIG. 4B is a graph showing the growth curves of TKp53^{L/L}p16^{L/L} melanoma cells with LKB1 knockdown treated with vehicle (shLkb1+DMSO, data indicated by squares) or 30 nM of the pan-SRC family kinase inhibitor dasatinib (shLkb1+Dasatinib, data indicated by “x”s) and of TKp53^{L/L}p16^{L/L} melanoma cells without Lkb1 knockdown treated with vehicle (NS+DMSO, data indicated by diamonds) or 30 nM dasatinib (NS+Dasatinib, data indicated by triangles). Cell numbers were counted at 0, 24, 48, 72, and 96 hours as indicated in the x axis.

FIG. 4C is a bar graph of closure index for TKp53^{L/L}p16^{L/L} melanoma cells with LKB1 knockdown treated with vehicle (shLkb1+DMSO) or 30 nM dasatinib (shLkb1+Dasatinib) and for TKp53^{L/L}p16^{L/L} melanoma cells without Lkb1 knockdown treated with vehicle (NS+DMSO) or 30 nM dasatinib (NS+Dasatinib). Closure index was measured 12 hours after wounding. The asterisks indicate a significant difference (P<0.05).

FIG. 4D is a graph of tumor invasiveness (quantified as number of invaded cells) measured by matrigel invasion assay for TKp53^{L/L}p16^{L/L} melanoma cells with LKB1 knockdown treated with vehicle (shLkb1+DMSO) or 30 nM dasatinib (shLkb1+Dasatinib) and for TKp53^{L/L}p16^{L/L} melanoma cells without Lkb1 knockdown treated with vehicle (NS+DMSO) or 30 nM dasatinib (NS+Dasatinib). Data is graphed as a mean of three replicates and standard deviation (SD) in all panels. The numbers above the panels indicate the ratio of number of invaded cells for vehicle treated cells vs number of invaded cells for dasatinib treated cells.

FIG. 5A is a pair of photographs of representative Western analyses of LKB1 and actin expression in A2058 human melanoma cells transduced with nonspecific short hairpin RNA (NS) or short hairpin RNA to LKB1 (shLKB1). “U” stands for untreated melanoma cells.

FIG. 5B is a bar graph of the tyrosine phosphorylation status of SRC family kinases (SFKs) members (LCK, LYN, SRC, YES, FGR, FYN, BLK, and HCK, from left to right as indicated under the x axis) in A2058 human melanoma cells with (shLKB1) or without (NS) LKB1 knockdown. MFI (mean fluorescence intensity) values of three replicates per kinase are shown. The asterisks indicate a significant difference (P<0.05). Data is graphed as the mean of three replicates and standard deviation (SD).

FIG. 5C is a series of photographs of representative Western analyses of A2058 human melanoma cells expressing short hairpin LKB1 (shLKB1) transfected with scrambled control (Control) short interfering RNA (siRNA) or siRNAs targeting SRC, FYN, or YES (i.e., SRC siRNA, FYN siRNA, and YES siRNA, from top to bottom). Cell lysates were immunoblotted with the antibodies indicated at the right of the photographs 48 hours after transfection. U=untreated.

FIG. 5D is a bar graph showing the close index results of an in vitro scratch assay of A2058 human melanoma cells with (shLKB1) or without (NS) LKB1 knockdown transfected with the indicated control or SRC family kinase (SFK) short interfering RNAs (siRNA; i.e., Control siRNA, SRC siRNA, FYN siRNA, or YES siRNA, from left to right). Cells were subjected to in vitro scratch assay 48 hours after siRNA transfection. Data is graphed as the mean of three replicates and standard deviation (SD).

FIG. 5E is a bar graph showing the results of a matrigel invasion assay of A2058 human melanoma cells with (shLKB1) or without (NS) LKB1 knockdown transfected with the indicated control or SRC family kinase (SFK) short interfering RNAs (siRNAs; i.e., Control siRNA, SRC siRNA, FYN siRNA, or YES siRNA, from left to right). Cells were subjected to matrigel invasion assay 48 hours after siRNA transfection. Tumor invasiveness data is graphed as the mean number of invaded cells of three replicates and standard deviation (SD).

FIG. 6A is a bar graph of Cd24 expression of melanoma cells with Lkb1 function (TKp53^{L/L}p16^{L/L} to and TRIA) and without Lkb1 function (TKLkb1^{L/L} and TKp53^{L/L}Lkb1^{L/L}) examined by flow cytometry.

FIG. 6B is a bar graph of Cd24 expression of isogenic melanoma cells with and without Lkb1 function examined by flow cytometry. TKp53^{L/L}Lkb1^{L/L} cells were transduced with non-functional Lkb1-KD (“kinase-dead”) or Lkb1. For comparison, Cd24 expression is also shown for TKp53L/LLkb1L/L cells transduces with a control vector (vector). TKp53^{L/L}p16^{L/L} cells were transduced with nonspecific short hairpin RNA (NS) or short hairpin RNA targeting Lkb1 (shLkb1).

FIG. 6C is a graph of the growth curves of Cd24⁺ (squares) and Cd24⁻ (diamonds) cells isolated from TKp53^{L/L}Lkb1^{L/L} melanoma cells by fluorescence-activated cell sorting (FACS). The data is graphed as the mean of three replicates and standard deviation.

FIG. 6D is a bar graph of mean close index of the Cd24⁺ and Cd24⁻ cells described in FIG. 6C subjected to scratch assay. The close index is graphed as the mean of three replicates and standard deviation (SD).

FIG. 6E is a bar graph of tumor invasiveness of the Cd24⁺ and Cd24⁻ cells described in FIG. 6C subjected to matrigel invasion assay. Tumor invasiveness is measured as the number of invaded cells and graphed as the mean of three replicates and standard deviation (SD).

FIG. 7A is a bar graph of CD24 expression in A2058 human melanoma cells with LKB1 knockdown (shLKB1) prepared by transduction with short hairpin LKB1 RNA. For comparison, CD24 expression is also provided for A2058 cells transduced with a nonspecific short hairpin RNA (NS) and for untreated A2058 cells (U).

FIG. 7B is a set of photographs of Western analyses of pan-SRC family kinase (p-SFK) expression CD24⁺ and CD24⁻ cells isolated by fluorescence-activated cell sorting (FACS) from A2058 cells expressing short hairpin RNA (shRNA) to LKB1 (shLKB1). For comparison, data for cells expression a non specific shRNA (NS) is also shown. Expression of p-SFK is compared to expression of actin.

FIG. 7C is a graph of CD24 mRNA expression in A2058 human melanoma cells with LKB1 knockdown (A2058+shLKB1) treated with 30 nM dasatinib (squares), 100 nM dasatinib (triangles), or vehicle (dimethyl sulfoxide, DMSO; diamonds), and harvested for analysis at 0, 12, 24, or 48 hours (h). The expression of mRNA was measured by quantitative reverse transcriptase polymerase chain reaction (RT-PCR) and calculated as relative expression to A2058 cells with non-specific (NS)-shRNA.

FIG. 7D is a graph of CD24 protein expression in A2058 human melanoma cells with LKB1 knockdown (A2058+shLKB1) treated with 30 nM dasatinib (squares), 100 nM dasatinib (triangles), or vehicle (dimethyl sulfoxide, DMSO; diamonds), and harvested for analysis at 0, 12, 24, 48 or 72 hours (h). Protein expression was measured by flow cytometry. N=3 replicates.

FIG. 7E is a bar graph showing CD24 expression in A2058 human melanoma cells with LKB1 knockdown (A2058+shLKB1) and transfected with SRC-family kinase (SKF) short interfering RNAs (siRNAs; SRC siRNA, FYN siRNA, and YES siRNA) or control siRNA. CD24 expression was measured by flow cytometry 72 hours after transfection. N=3 replicates. Error bars show standard deviation (SD).

FIG. 8A is a bar graph showing colony forming efficiencies of Cd24⁺ and Cd24⁻ cells from Lkb1-competent and Lkb1-deficient cell lines of TKp53^{L/L}Lkb1^{L/L} and TKp53^{L/L}p16^{L/L} genotypes. The colony forming efficiencies were measured by plating a single cell per well of the indicated genotypes. Colony forming cells were counted for each 96 well plate. The data is graphed as mean of at least three replicates and standard deviation (SD).

FIG. 8B is a bar graph of mean tumor volume of tumors generated by isolating Cd24⁺ and Cd24⁻ cells from Lkb1-competent and Lkb1-deficient cell lines of TKp53^{L/L}Lkb1^{L/L} and TKp53^{L/L}p16^{L/L} genotypes by fluorescence-activated cell sorting (FACS) and injecting the cells into the ears of nude mice. N=5 for each group. P-values were determined by two-tailed t-test.

### BRIEF DESCRIPTION OF THE SEQUENCE LISTING

Each of the sequences listed in Table 1, including the annotations and references cited in the corresponding GENBANK® Accession Nos., is incorporated herein by reference in its entirety.

The Sequence Listing is provided herewith as an ASCII.txt file entitled 421_{—}298.ST25, created Feb. 4, 2013, 3400 bytes (34 kilobytes), and is incorporated here by reference in its entirety.

TABLE 1
Listing of GENBANK ® Accession Numbers for Nucleic Acid
and Amino Acid Sequences of Exemplary Gene Products

| | Exemplary Nucleotide | | Exemplary Amino Acid | |
|---|---|---|---|---|
| | Sequence | | Sequence | |
| | GENBANK ® | SEQ | GENBANK ® | SEQ |
| Description | Accession No. | ID NO: | Accession No. | ID NO: |
| Human LKB1 | NM_000455 | 1 | NP_000446 | 2 |
| Human YES | NM_005433 | 3 | NP_005424 | 4 |
| Human CD24 | NM_013230 | 5 | NP_037362 | 6 |

### DETAILED DESCRIPTION

The present subject matter will be now be described more fully hereinafter with reference to the accompanying Examples, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### I. GENERAL CONSIDERATIONS

The LKB1 (or STK11) gene encodes a CAMK family serine/threonine kinase which phosphorylates and activates a number of conserved targets, including 5′-adenosine monophosphate-activated protein kinase (AMPK) and the AMPK-related kinases. See Alessi et al., 2006. Germline mutations in LKB1 (STK11) are associated with the Peutz-Jeghers syndrome (PJS; see Hemminki et al., 1998; and Jenne et al., 1998), and autosomal, dominant disorder characterized by hamartomatous polyps of the gastrointestinal tract, increased mucocutaneous pigmentation, and increased cancer risk. See Giardiello et al., 2000; Giardiello et al., 1987; Jeghers et al., 1949; and Lim et al., 2004. Although most commonly associated with cancers of gastrointestinal origin, PJS patients also demonstrate an increased risk of developing non-GI cancer (e.g., of the breast, ovary and testis). See Lim et al., 2004; and Sanchez-Cespedes, 2007. In addition, somatic LKB1 mutations occur in several types of sporadic cancers, including in 10% of cutaneous melanoma. See Forbes et al., 2011; Guldberg et al., 1999; and Rowan et al., 1999.

A role for LKB1 in regulating tumor differentiation and metastasis has been suggested in epithelial cancers. For example, somatic inactivation of Lkb1 combined with activation of K-Ras in genetically engineered murine models (GEMMs) of lung cancer results in tumors with an expanded spectrum of tumor differentiation and considerably augmented metastasis compared to K-Ras-driven tumors lacking p53 or Ink4a/Arf. See Ji et al., 2007. See also, U.S. Patent Application Publication No. 2011/0119776; incorporated herein by reference in its entirety. LKB1 mutation is associated with advanced stage and metastasis in human patients with aerodigestive carcinomas. See Guervos et al., 2007; and Matsumoto et al., 2007. Loss of LKB1 has also been reported to promote several metastatic behaviors (e.g. epithelial-mesenchymal transition (EMT), resistance to anoikis, increased motility and invasiveness) in a variety of epithelial cell types in vitro through diverse mechanisms including inhibition of SIK1 (see Cheng et al., 2009) or AMPK (See Taliaferro-Smith et al., 2009) as well as activation of EMT, focal adhesion, and SRC-Family Kinases (SFKs). See Carretero et al., 2010.

While attenuation of LKB1 appears to occur in a human melanoma via either direct genetic inactivation or indirect functional inhibition (e.g. through mutation of upstream regulators such as B-RAF), to date there has been almost no study of the impact of LKB1 loss on melanomagenesis. Using melanocyte-specific genetically engineered murine models (GEMMs), the presently disclosed subject matter shows that Lkb1 loss leads to a 100% penetrance of metastatic melanoma. This enhancement of metastasis appears to require activation of the YES SRC-family kinase to augment a rare, pro-metastatic CD24⁺ tumor sub-fraction with properties of tumor stem cells. The presently disclosed subject matter provides new data related to how LKB1 loss promotes metastasis in a wide variety of cancers, and identifies new therapeutic targets in melanoma.

More particularly, as described herein, by somatically inactivating Lkb1 with K-Ras activation (+/−p53 loss) in murine melanocytes, variably pigmented and highly metastatic melanoma with 100% penetrance are observed. LKB1 deficiency results in increased phosphorylation of the SRC-family kinase (SFK) YES and the subsequent expansion of a CD24⁺ cell population which shows increased metastatic behavior in vitro and in vivo relative to isogenic CD24⁻ cells. Without being bound to any one theory, these results suggest that LKB1 inactivation in the context of RAS activation facilitates metastasis by inducing a SFK-dependent expansion of a pro-metastatic, CD24⁺ tumor sub-population.

Metastatic melanoma has a poor prognosis. Predicting prognosis in the disease can play a role in determining patient therapy. Additionally, determination of somatic mutations in a tumor can guide choice of therapy (e.g EGFR mutation in lung cancer). Melanoma is treatment refractory, and new therapeutic approaches are needed in melanoma.

Thus, in some embodiments the presently disclosed subject matter provides for (1) the use of LKB1 mutation status or expression to predict melanoma prognosis and response to therapeutics; (2) the use of YES expression and phosphorylation level to predict melanoma prognosis and response to therapeutics; (3) the use of CD24 expression to predict melanoma prognosis and response to therapeutics; and/or (4) the use of targeted inhibitors of SRC family kinases (especially YES) to treat melanoma. To elaborate, the loss of LKB1 in melanoma models promotes widespread and high-grade metastasis. This enhancement of metastasis requires activation of YES kinase, a SRC-family member. LKB1 inactivation leads to the expansion of a pro-metastatic CD24⁺ tumor subfraction. YES and CD24 are therapeutic targets in LKB1-deficient melanoma.

### II. DEFINITIONS

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” mean “one or more” when used in this application, including the claims. Thus, the phrase “a cell” refers to one or more cells, unless the context clearly indicates otherwise.

Throughout the specification and claims, a given chemical formula or name shall encompass all optical isomers and stereoisomers, as well as racemic mixtures where such isomers and mixtures exist.

Unless otherwise indicated, all numbers expressing quantities of ingredients, reaction conditions, and so forth used in the specification of the presently disclosed subject matter are to be understood as being modified in all instances by the term “about”. The term “about”, as used herein when referring to a measurable value such as an amount of mass, weight, time, volume, temperature, pressure, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods. Accordingly, unless indicated to the contrary, the numerical parameters set forth in this specification of the presently disclosed subject matter are approximations that can vary depending upon the desired properties sought to be obtained by the presently disclosed subject matter.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including,” “containing,” or “characterized by” is inclusive or open-ended and does not exclude additional, unrecited elements or method steps. “Comprising” is a term of art used in claim language which means that the named elements are present, but other elements can be added and still form a construct or method within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Ayes (birds), and Mammalia (mammals)), and all Orders and Families encompassed therein.

Similarly, all genes, gene names, and gene products disclosed herein are intended to correspond to orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, the genes and/or gene products disclosed herein are intended to encompass homologous genes and gene products from other animals including, but not limited to other mammals, fish, amphibians, reptiles, and birds.

The methods and compositions of the presently disclosed subject matter are particularly useful for warm-blooded vertebrates. Thus, the presently disclosed subject matter concerns mammals and birds. More particularly provided is the use of the methods and compositions of the presently disclosed subject matter on mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos or as pets, as well as fowl, and more particularly domesticated fowl, e.g., poultry, such as turkeys, chickens, ducks, geese, guinea fowl, and the like, as they are also of economic importance to humans. Thus, also provided is the application of the methods and compositions of the presently disclosed subject matter to livestock, including but not limited to domesticated swine (pigs and hogs), ruminants, horses, poultry, and the like.

As used herein the term “gene” refers to a hereditary unit including a sequence of DNA that occupies a specific location on a chromosome and that contains the genetic instruction for a particular characteristic or trait in an organism. Similarly, the phrase “gene product” refers to biological molecules that are the transcription and/or translation products of genes. Exemplary gene products include, but are not limited to mRNAs and peptides or polypeptides that result from translation of mRNAs. Any of these naturally occurring gene products can also be manipulated in vivo or in vitro using well known techniques, and the manipulated derivatives can also be gene products. For example, a cDNA is an enzymatically produced derivative of an RNA molecule (e.g., an mRNA), and a cDNA is considered a gene product. Additionally, peptide or polypeptide translation products of mRNAs can be enzymatically fragmented using techniques well know to those of skill in the art, and these peptide or polypeptide fragments are also considered gene products.

As used herein, the term “LKB1” refers to the LKB1 gene or gene product. Exemplary LKB1 gene sequences and products from humans are described in GENBANK® Accession No. NM_{—}000455. Gene synonyms include STK11, hLKB1, and PJS.

As used herein, the term “YES” refers to the YES gene or gene product. Exemplary YES gene sequences and products from humans are described in GENBANK® Accession No. NM_{—}005433. Gene synonyms include YES1, c-yes, HsT441, P61-YES and Yes.

As used herein, the term “CD24” refers to the CD24 gene or gene product. Exemplary CD24 gene sequences and products are described in GENBANK® Accession No. NM_{—}013230. Gene synonyms include CD24A. HSA (for “heat stable antigen”), “CD24a” and “Nectadrin” have also been used in the literature as synonyms for CD24.

It is understood that while the nucleotide and amino acid sequences for the human orthologs of LKB1, YES, and CD24 are disclosed herein, orthologs of these genes from other species are also included within the presently disclosed subject matter.

The term “isolated”, as used in the context of a nucleic acid or polypeptide (including, for example, a peptide), indicates that the nucleic acid or polypeptide exists apart from its native environment. An isolated nucleic acid or polypeptide can exist in a purified form or can exist in a non-native environment.

The terms “nucleic acid molecule” and “nucleic acid” refer to deoxyribonucleotides, ribonucleotides, and polymers thereof, in single-stranded or double-stranded form. Unless specifically limited, the term encompasses nucleic acids containing known analogues of natural nucleotides that have similar properties as the reference natural nucleic acid. The terms “nucleic acid molecule” and “nucleic acid” can also be used in place of “gene”, “cDNA”, and “mRNA”. Nucleic acids can be synthesized, or can be derived from any biological source, including any organism.

The term “isolated”, as used for example in the context of a cell, nucleic acid, or peptide, indicates that the cell, nucleic acid, or peptide exists apart from its native environment. In some embodiments, “isolated” refers to a physical isolation, meaning that the cell, nucleic acid or peptide has been removed from its native environment (e.g., from a subject).

As used herein, the terms “peptide” and “polypeptide” refer to polymers of at least two amino acids linked by peptide bonds. Typically, “peptides” are shorter than “polypeptides”, but unless the context specifically requires, these terms are used interchangeably herein. In some embodiments, peptides can refer to polymers of between 2 and 20, 30, 40, or 50 amino acids. In some embodiments, polypeptides can refer to polymers of more than 20, 30, 40, or 50 amino acids.

The terms “presence” or “absence” can refer to a situation where a mutation of a gene is present or absent, can refer to the situation where expression of the gene is present or absent in a given sample, can refer to the situation wherein activity of a gene product is present or absent, and/or wherein activation (e.g. phosphorylation) of a gene product is present or absent. With regard to expression levels, expression levels can be compared to a typical basal level of expression of a particular gene or gene product in a given context, or to another standard. Similarly, a phosphorylation level can refers to a level of phosphorylation of a particular gene or gene product and can be compared to a basal level or normal tissue level of phosphorylation, in some embodiments. In some cases an expression level or phosphorylation level can be substantially zero, and in this case the level can be referred to as absent. In some cases the presence of any level of expression, activation, and/or activity of a particular gene or gene product can be used in accordance with the presently disclosed subject matter.

In some embodiments, the “mutation” of the presently disclosed subject matter is a mutation that results in decreased gene expression (e.g. decreased LKB1 protein abundance), decreased activity of a gene product, or both. Thus, for example, in some embodiments of the presently disclosed subject matter, the presence or absence of an inactivating LKB1 mutation is detected, wherein the inactivating LKB1 mutation is a mutation that results in decreased LKB1 expression, decreased LKB1 activity, or both.

Inactivating mutations can cause frameshifts, premature stop codons, and in-frame deletions of coding material. Inactivation mutations can affect RNA splicing to lead to decreased LKB1 protein production, or can affect LKB1 mRNA expression (for example, by altering the LKB1 promoter or other cis-regulatory elements).

In some embodiments, the mutation is a large deletion of chromosome 19p13, where LKB1/STK11 resides. These large deletions can span the entire chromosome or an arm of the chromosome. In some embodiments, the mutation can be smaller, e.g., a deletion of less than 1,000 base pairs. For example, the smaller deletion can target one or only a few exons of LKB1 or can be a deletion of the promoter of LKB1 that does not target the coding sequence of LKB1.

In addition to larger deletions, the inactivating mutations can be point mutations and small insertion/deletion mutations. The inactivating mutations can be nonsense mutations or missense mutations.

Table 2 provides some exemplary mutations of LKB1 affecting the coding sequence. The mutations in Table 2 are derived from the Catalog of Somatic Mutations in Cancer (COSMIC or COSM), available online on the website for the Welcome Trust Sanger Institute. The numbers in the Mutation ID column of Table 2 refer to COSMIC accession/identification numbers. The numbers in the Coding Sequence (CDS) mutation column refer to the nucleotide position in the open reading frame of SEQ ID NO: 1, which starts at nucleotide number 1116 of SEQ ID NO: 1 and ends at nucleotide number 2417 of SEQ ID NO: 1. Thus, for example, the first entry in Table 2 refers to the substitution of a C for the T at position number 2 of the open reading frame of SEQ ID NO: 1 (i.e., at nucleotide 1117 of SEQ ID NO: 1). The second entry in Table 2 refers to the substitution of a A for a C at position number 17 of the open reading frame of SEQ ID NO: 1 (i.e., at nucleotide 1132 of SEQ ID NO: 1). The numbers in the Position and Amino Acid (AA) mutation columns refer to the amino acid position in SEQ ID NO: 2. Thus, for example, the first entry in Table 1 refers to a mutation related to the first amino acid in SEQ ID NO: 2, while the second entry refers to a mutation related to the sixth amino acid in SEQ ID NO: 2.

TABLE 2
Exemplary LKB1 Mutations.

| | | | Mutation ID | |
|---|---|---|---|---|
| Position | CDS Mutation | AA Mutation | (COSM) | Type |
| 1 | c.2T>C | p.M1T | 20951 | substitution_missense |
| 6 | c.17C>A | p.P6Q | 29463 | substitution_missense |
| 14 | c.40G>A | p.E14K | 21385 | substitution_missense |
| 19 | c.56C>A | p.S19* | 29462 | substitution_nonsense |
| 33 | c.97G>T | p.E33* | 95668 | substitution_nonsense |
| 36 | c.108C>A | p.Y36* | 20947 | substitution_nonsense |
| 37 | c.110A>T | p.Q37L | 48783 | substitution_missense |
| 37 | c.109C>T | p.Q37* | 12925 | substitution_nonsense |
| 44 | c.130A>T | p.K44* | 20868 | substitution_nonsense |
| 50 | c.148_159del12 | p.L50_D53del | 51519 | deletion_inframe |
| 53 | c.157delG | p.D53fs*11 | 48969 | deletion_frameshift |
| 56 | c.166_178del13 | p.G56fs*4 | 48970 | deletion_frameshift |
| 56 | c.166G>T | p.G56W | 48784 | substitution_missense |
| 57 | c.167_168insTTCC | p.E57fs*107 | 166199 | insertion_frameshift |
| 57 | c.169G>T | p.E57* | 29464 | substitution_nonsense |
| 57 | c.169delG | p.E57fs*7 | 21212 | deletion_frameshift |
| 60 | c.180C>G | p.Y60* | 20874 | substitution_nonsense |
| 60 | c.? | p.Y60* | 133062 | substitution_nonsense |
| 60 | c.180C>A | p.Y60* | 48900 | substitution_nonsense |
| 60 | c.180delC | p.Y60fs*1 | 27322 | deletion_frameshift |
| 65 | c.193G>T | p.E65* | 20876 | substitution_nonsense |
| 66 | c.196G>A | p.V66M | 21384 | substitution_missense |
| 70 | c.208G>T | p.E70* | 25846 | substitution_nonsense |
| 78 | c.232A>G | p.K78E | 48785 | substitution_missense |
| 86 | C.256C>G | p.R86G | 29006 | substitution_missense |
| 87 | c.260G>A | p.R87K | 21075 | substitution_missense |
| 91 | c.271_272GG>TT | p.G91L | 48913 | substitution_missense |
| 100 | c.? | p.Q100E | 34162 | substitution_missense |
| 107 | c.320A>G | p.H107R | 29465 | substitution_missense |
| 108 | c.322A>T | p.K108* | 564718 | substitution_nonsense |
| 120 | c.358G>T | p.E120* | 20875 | substitution_nonsense |
| 137 | c.411_412GG>TT | p.Q137_E138>H* | 1141538 | complex |
| 137 | c.409C>T | p.Q137* | 48901 | substitution_nonsense |
| 144 | c.431delC | p.P144fs*17 | 48971 | deletion_frameshift |
| 152 | c.454C>T | p.Q152* | 96526 | substitution_nonsense |
| 159 | c.475C>T | p.Q159* | 27316 | substitution_nonsense |
| 160 | c.479T>C | p.L160P | 21382 | substitution_missense |
| 163 | c.488G>A | p.G163D | 21352 | substitution_missense |
| 165 | c.493G>T | p.E165* | 48902 | substitution_nonsense |
| 168 | c.503A>G | p.H168R | 564715 | substitution_missense |
| 170 | c.508C>T | p.Q170* | 20943 | substitution_nonsense |
| 171 | c.511G>A | p.G171S | 21354 | substitution_missense |
| 176 | c.527A>C | p.D176A | 564714 | substitution_missense |
| 179 | c.536C>T | p.P179L | 51520 | substitution_missense |
| 179 | c.535C>T | p.P179S | 238600 | substitution_missense |
| 180 | c.539G>T | p.G180V | 96527 | substitution_missense |
| 181 | c.541A>T | p.N181Y | 564713 | substitution_missense |
| 181 | c.542A>T | p.N181I | 564712 | substitution_missense |
| 191 | c.571A>T | p.K191* | 48903 | substitution_nonsense |
| 194 | c.579delC | p.D194fs*93 | 48972 | deletion_frameshift |
| 194 | c.581A>T | p.D194V | 20957 | substitution_missense |
| 194 | c.580G>T | p.D194Y | 20944 | substitution_missense |
| 196 | c.587G>T | p.G196V | 48786 | substitution_missense |
| 199 | c.595G>A | p.E199K | 21359 | substitution_missense |
| 199 | c.595G>T | p.E199* | 25229 | substitution_nonsense |
| 205 | c.613G>A | p.A205T | 20953 | substitution_missense |
| 208 | c.622G>A | p.D208N | 21356 | substitution_missense |
| 210 | c.630C>A | p.C210* | 20869 | substitution_nonsense |
| 215 | c.644G>A | p.G215D | 21357 | substitution_missense |
| 216 | c.646T>C | p.S216P | 96336 | substitution_missense |
| 216 | c.647C>T | p.S216F | 25844 | substitution_missense |
| 217 | c.650delC | p.P217fs*70 | 20880 | deletion_frameshift |
| 218 | c.650_651insC | p.A218fs*48 | 20858 | insertion_frameshift |
| 220 | c.658C>T | p.Q220* | 13480 | substitution_nonsense |
| 223 | c.? | p.E223L | 133061 | substitution_missense |
| 223 | c.667G>T | p.E223* | 20870 | substitution_nonsense |
| 231 | c.691T>C | p.F231L | 21383 | substitution_missense |
| 235 | c.703A>T | p.K235* | 564711 | substitution_nonsense |
| 237 | c.709G>T | p.D237Y | 48787 | substitution_missense |
| 237 | c.709_709delG | p.D237fs*50 | 96530 | deletion_frameshift |
| 239 | c.717G>T | p.W239C | 333593 | substitution_missense |
| 242 | c.725G>T | p.G242V | 48788 | substitution_missense |
| 242 | c.724G>T | p.G242W | 564710 | substitution_missense |
| 251 | c.751G>C | p.G251R | 564708 | substitution_missense |
| 251 | c.752G>T | p.G251V | 564707 | substitution_missense |
| 264 | c.787_790delTTGT | p.F264fs*22 | 20857 | deletion_frameshift |
| 271 | c.810delG | p.S271fs*16 | 48973 | deletion_frameshift |
| 279 | c.835_836GG>TT | p.G279F | 85760 | substitution_missense |
| 281 | c.837delC | p.P281fs*6 | 20871 | deletion_frameshift |
| 281 | c.842delC | p.P281fs*6 | 12924 | deletion_frameshift |
| 282 | c.842_843insC | p.L282fs*3 | 25851 | insertion_frameshift |
| 294 | c.879_880insA | p.P294fs*24 | 29466 | insertion_frameshift |
| 297 | c.891G>C | p.R297S | 96528 | substitution_missense |
| 304 | c.910C>G | p.R304G | 48789 | substitution_missense |
| 304 | c.910C>T | p.R304W | 29468 | substitution_missense |
| 308 | c.923G>T | p.W308L | 26041 | substitution_missense |
| 312 | c.936delA | p.K312fs*24 | 20948 | deletion_frameshift |
| 314 | c.941C>A | p.P314H | 21353 | substitution_missense |
| 320 | c.957_958AG>T | p.V320fs*16 | 20958 | complex |
| 324 | c.971C>T | p.P324L | 21380 | substitution_missense |
| 327 | c.979_980insAG | p.D327fs*10 | 48942 | insertion_frameshift |
| 332 | c.996G>A | p.W332* | 18652 | substitution_nonsense |
| 367 | c.1100C>T | p.T367M | 21358 | substitution_missense |
| 389 | c.1165G>A | p.A389T | 48790 | substitution_missense |
| | c.?_?insG | p.?fs | 20877 | insertion_frameshift |

In some embodiments, the inactivating mutation can be a single-copy or two-copy mutation or deletion. Thus, the mutation can be inactivating even if it only causes haploinsufficiency. Accordingly, the melanoma can have a homozygous deletion mutation of LKB1, a deletion mutation of one allele and a point mutation of another allele of LKB1, or heterozygous mutations of LKB1. In some embodiments, the inactivating mutation can result in an amino acid substitution or deletion in a gene product.

In some embodiments of the presently disclosed subject matter, a profile can be created once an expression level is determined for a gene. As used herein, the term “profile” (e.g., a “gene expression profile”) refers to a repository of the expression level data that can be used to compare the expression levels of different genes among various subjects. For example, for a given subject, the term “profile” can encompass the expression levels of one or more of the genes disclosed herein detected in whatever units are chosen. The term “profile” is also intended to encompass manipulations of the expression level data derived from a subject. For example, once relative expression levels are determined for a given set of genes in a subject, the relative expression levels for that subject can be compared to a standard to determine if the expression levels in that subject are higher or lower than for the same genes in the standard. Standards can include any data deemed to be relevant for comparison.

As such, the presently disclosed methods can employ various techniques to generate the gene expression profiles required for the comparisons. See e.g., PCT International Patent Application Publication Nos. WO 2004/046098; WO 2004/110244; WO 2006/089268; WO 2007/001324; WO 2007/056332; WO 2007/070252, each of which is incorporated herein by reference in its entirety.

As used herein, a cell, nucleic acid, or peptide exists in a “purified form” when it has been isolated away from some, most, or all components that are present in its native environment, but also when the proportion of that cell, nucleic acid, or peptide in a preparation is greater than would be found in its native environment. As such, “purified” can refer to cells, nucleic acids, and peptides that are free of all components with which they are naturally found in a subject, or are free from just a proportion thereof.

### III. METHODS FOR PREDICTING MELANOMA PROGNOSIS

Provided in accordance with the presently disclosed subject matter are methods of predicting a melanoma prognosis. In some embodiments, the methods comprise:

(a) detecting one or more of the following in a biological sample comprising melanoma cells obtained from a melanoma of a subject:
(i) the presence or absence of a LKB1 mutation, or a LKB1 expression level;
(ii) a YES expression level, a YES phosphorylation level, or both; and
(iii) a CD24 expression level; and

(b) predicting a melanoma prognosis based on the detecting of step (a).

In some embodiments, the presence of an LKB1 mutation or of a reduced level of expression of LKB1 is indicative of a negative prognosis, e.g., is indicative that the melanoma is aggressive. “Aggressive” can refer to a metastatic (i.e., quickly growing and spreading) melanoma. As disclosed herein when reference is made to expression of LKB1, YES or CD24, it is generally meant to refer to expression of nucleic acid (e.g. mRNA) or protein. In some embodiments, the absence of an LKB1 mutation or the absence of a reduced level of expression of LKB1 is indicative of a positive prognosis, i.e., is indicative that the melanoma is non-aggressive (i.e., not metastatic). In some embodiments, an elevated level of expression of YES, elevated YES phosphorylation, or both, is indicative of a negative prognosis. In some embodiments, the absence of an elevated level of expression of YES, YES phosphorylation, or both, is indicative of a positive prognosis. In some embodiments, an elevated level of CD24 expression is indicative of a negative prognosis. In some embodiments, the absence of an elevated level of CD24 expression is indicative of a positive prognosis.

In some embodiments, an expression level is determined by a polymerase chain reaction (PCR)-based method, a microarray based method, or an antibody-based method. In some embodiments, an expression level is normalized relative to an expression level of one or more reference genes. In some embodiments, the expression level is compared to a standard. In some embodiments, the methods comprise determining an expression level for one or more genes selected from the group consisting of LKB1, YES, and CD24 in a biological sample comprising melanoma cells obtained from subject; and comparing the expression levels determined to a standard.

In some embodiments, the method further comprises assessing a risk of an adverse outcome of a subject with melanoma. In some embodiments, the adverse outcome includes, but is not limited to, decreased Overall Survival (OS) and/or Disease-Free Survival (DFS)) that would occur in a subject relative to other subjects with melanoma.

### IV. METHODS FOR PREDICTING A RESPONSE TO THERAPY

Provided here in accordance with the presently disclosed subject matter are methods of predicting a response to a therapy by a melanoma in a subject having the melanoma and receiving the therapy. In some embodiments, the methods comprise:

(a) detecting one or more of the following in a biological sample comprising melanoma cells obtained from a melanoma of a subject:
(i) the presence or absence of a LKB1 mutation, or a LKB1 expression level;
(ii) a YES expression level, a YES phosphorylation level, or both; and
(iii) a CD24 expression level; and

(b) predicting a response to the therapy based on the detecting of step (a). The therapy or treatment can selected from the group comprising but not limited to surgical resection of the melanoma, chemotherapy, molecular targeted therapy, immunotherapy, and combinations thereof.

In some embodiments, the presence of an LKB1 mutation or of a reduced level of expression of LKB1 is indicative of a resistance to the therapy. As disclosed herein when reference is made to expression of LKB1, YES or CD24, it is generally meant to refer to expression of nucleic acid (e.g. mRNA) or protein. In some embodiments, the absence of an LKB1 mutation or of a reduced level of expression of LKB1 is indicative of a lack of a resistance to the therapy. In some embodiments, an elevated level of YES expression, YES phosphorylation, or both, is indicative of a resistance to the therapy. In some embodiments, the absence of an elevated level of YES expression, YES phosphorylation, or both, is indicative of a lack of a resistance to the therapy. In some embodiments, an elevated level of CD24 expression is indicative of a resistance to the therapy. In some embodiments, the absence of an elevated level of CD24 expression is indicative of a lack of resistance to the therapy.

In some embodiments, an expression level is determined by a PCR-based method, a microarray based method, or an antibody-based method. In some embodiments, an expression level is normalized relative to an expression level of one or more reference genes. In some embodiments, the expression level is compared to a standard. In some embodiments, the methods comprise (a) determining the expression level of one or more genes selected from the group consisting of LKB1, YES, and CD24 in a biological sample comprising melanoma cells obtained from the melanoma of the subject; and (b) comparing the expression levels determined to a standard.

The presently disclosed subject matter also provides methods for predicting a clinical outcome of a treatment in a subject diagnosed with melanoma. In some embodiments, the methods comprise (a) determining the expression level of one or more genes selected from the group consisting of LKB1, YES, and CD24 in a biological sample comprising melanoma cells obtained from the melanoma of the subject; and (b) comparing the expression levels determined to a standard, wherein the comparing is predictive of the clinical outcome of the treatment in the subject.

As used herein, the phrase “clinical outcome” refers to any measure by which a treatment designed to treat melanoma can be measured. Exemplary clinical outcomes include Recurrence-Free Interval (RFI), Overall Survival (OS), Disease-Free Survival (DFS), or Distant Recurrence-Free Interval (DRFI).

### V. METHODS FOR MANAGING TREATMENT

Provided here in accordance with the presently disclosed subject matter are methods for managing treatment of a subject with melanoma. In some embodiments, the methods comprise:

(a) detecting one or more of the following in a biological sample comprising melanoma cells obtained from a melanoma of a subject:
(i) the presence or absence of a LKB1 mutation, or a LKB1 expression level;
(ii) a YES expression level, a YES phosphorylation level, or both; and
(iii) a CD24 expression level; and

(b) managing treatment of the subject based on the detecting of step (a).

In the context of the presently disclosed subject matter, the term “managing treatment” can refer to choices made in selecting treatment options for a subject having melanoma. In some embodiments, the detecting of step (a) can suggest an altered treatment choice (i.e., changing the type or amount of treatment the subject is receiving). Depending on the evaluations made in accordance with the presently disclosed subject matter, the altered treatments can be aggressive treatment choices or conservative treatment choices. An aggressive treatment choice is a choice made based at least in part on the perceived likelihood that the melanoma will metastasize. An aggressive treatment choice can include increasing the dose of a therapeutic agent, adding an additional treatment to the current treatment regime, or using a more severe or radical treatment choice. Conversely, a conservative treatment choice is a choice made when after an evaluation of in accordance with the presently disclosed subject matter, a less severe or radical treatment choice is made (as compared to an aggressive choice), based at least in part on the perceived likelihood that the melanoma will not metastatsize. Depending on the evaluation of a particular subject, aggressive or conservative choices can include: surgical resection of the melanoma, chemotherapy (including but not limited employing combinations of chemotherapeutic agents and employing in treatment of the melanoma a chemotherapeutic agent approved for treating another type of cancer), molecular targeted therapy, immunotherapy, other experimental therapy, and combinations thereof. The choice of whether or not to pursue “adjuvant” chemotherapy or immunotherapy can be based on status of LKB1, YES, or CD24. Adjuvant therapy refers to the practice of treating patients who have no overt evidence of disease (e.g. after surgical resection) to prevent disease relapse at a later time point. In patients that have no evidence of disease, adjuvant treatment is an aggressive course.

In some embodiments, the presence of an LKB1 mutation or of a reduced level of expression of LKB1 is indicative of the need or option for pursuing an aggressive treatment choice. As disclosed herein when reference is made to expression of LKB1, YES or CD24, it is generally meant to refer to expression of nucleic acid (e.g. mRNA) or protein. As disclosed herein when reference is made to expression of LKB1, YES or CD24, it is generally meant to refer to expression of nucleic acid (e.g. mRNA) or protein. In some embodiments, the absence of an LKB1 mutation or of a reduced level of expression of LKB1 is indicative of the need or option for pursuing for a conservative treatment choice. In some embodiments, an elevated level of YES expression, YES phosphorylation, or both, is indicative of the need or option for pursuing an aggressive treatment choice. In some embodiments, the absence of an elevated level of YES expression, YES phosphorylation, or both, is indicative of the need or option for pursuing a conservative treatment choice. In some embodiments, an elevated level of CD24 expression is indicative of the need or option for pursuing an aggressive treatment choice. In some embodiments, the absence of an elevated level of CD24 expression is indicative of the need or option for pursuing a conservative treatment choice. That is, in any or all of the foregoing embodiments, a physician or other health care professional can suggest to the subject an aggressive or a conservative approach to therapy, based on the mentioned evaluations.

### VI. METHODS OF TREATING MELANOMA

Methods of treating melanoma in a subject in need thereof are also provided herein. In some embodiments, the methods comprise administering to the subject an effective amount of an inhibitor of a SRC family kinase to treat a melanoma in the subject. The inhibitor can be administered alone or in combination with another therapeutic agent (such as but not limited to those listed in Table 3). In some embodiments, a targeted inhibitor of a SRC family kinase (i.e., an inhibitor of a particular SRC family kinase) is administered. In some embodiments, a targeted inhibitor of YES is administered. In some embodiments, the subject is a mammal.

Representative clinically studied SRC inhibitors include dasatinib (Bristol-Myers-Squibb, FDA approved for imitinib-resistant CML), saracatinib (AZD0530, Astra Zeneca), bosutinib (SKI-606, Wyeth), KX2-391 (KX01, Kinex), XL228, AZM475271, XL999, SU6656 (the clinical trial status of this compound in ClinicalTrials.gov is not clear).

Representative preclinical SRC inhibitors include PP1 (not presently viewed as suitable for clinical use), PP2 (not presently viewed as suitable for clinical use), AP23846 (Ariad), Herbimycin A (benzochinoid antibiotic related to geldanamycin), CGP76030 (Novartis), 1I (Nbenzyl-1-(2-chloro-2-phenylethyl)-1H-pyrazolo[3,4-d]pyrimidin-4-amine, and 7-(2,6-dichlorophenyl)-5-methylbenzo[4-(2-pyrrolidin-1-ylethoxy)phenyl]-amine (TargeGen, WuXi PharmaTech).

TABLE 3
SRC Inhibitors with Other Agents in Clinical Trials

| | | | Combination |
|---|---|---|---|
| Drug | Phase | Tumor type | agent |
| Dasatinib | II | Advanced-NSCLC/Colorectal/ | — |
| | | Pancreatic/HNSCC/Breast/ | |
| | | SCLC/Melanoma | |
| | II | Resectable NSCLC/HNSCC | Erlotinib |
| | I-II | Advanced NSCLC | Erlotinib |
| | I | Breast | Capecitabine |
| | I | Breast | Paclitaxel |
| | I-II | Prostate/Castration | Docetaxel |
| | | resistant prostate cancer | |
| | I | Colon | FOLFOX6/ |
| | | | Cetuximab |
| Saracatinib | II | Prostate/Pancreatic/ | — |
| | | Osteosarcoma/Soft | |
| | | tissuesarcoma/Melanoma/ | |
| | | Gastration-resistant | |
| | | prostate cancer/Thymoma/ | |
| | | Colorectal/HNSCC | |
| | II | Advanced NSCLC/SCLC | |
| | | Carboplatin/Paclitaxel | |
| | I | Advanced solid tumor | Cediranib |
| | I-II | Pancreatic | Gemcitabine |
| | II | Ovarian | Carboplatin |
| | II | Prostate/Breast with | Zoledronic |
| | | bone metastasis | acid |
| Bosutinib | II | Breast | — |
| | II | Breast | Exemestane |
| | II | Breast | Letrozole/ |
| | | | Capecitabine |
| | I-II | Advanced solid tumor | Capecitabine |
| XL228 | I | Advanced solid tumor | — |
| KX2-391 | I | Advanced solid tumor/ | — |
| | | Lymphoma | |
| AZM475271 | I-II | Pancreatic | — |
| XL999 | I | Advanced solid tumor | — |

Also disclosed herein in some embodiments are methods of treating melanoma in a subject in need thereof, comprising providing a subject suffering from a melanoma wherein LKB1, YES and/or CD24 status for the subject's melanoma has been assessed; and administering to the subject an effective amount of a therapeutic agent to treat the melanoma in the subject based on the status of LKB1, YES and/or CD24 of the subject's melanoma (such as a tumor). Representative therapeutic agents that can be employed (for example, chosen or excluded based on LKB1, YES and/or CD24 status) include, but are not limited to, rapamycin, rapamycin analogues, RAD001, metformin and related molecules, PI3K inhibitors (BEZ235), RAF inhibitors (vemurafinib), MEK and ERK inhibitors (e.g. AZD6224), CD24 antibodies (including CD24 monoclonal antibodies), CDK inhibitors, interferon's (such as IFN-alpha), ipilimumab, other forms of immunotherapy, anti-angiogenesis agents (e.g. avastin), cytotoxic chemotherapies (e.g. cyclophosphamide, paclitaxel, doxorubicin) or any combination of the foregoing agents. Indeed the use of any therapeutic agent whose use could be predicated on LKB1, YES, and/or CD24 status is provided in accordance with the presently disclosed subject matter. In some embodiments, LKB1, YES and CD24 status can be determined by mutational testing (e.g. sequencing of tumor DNA or RNA) and/or expression analysis (e.g. to tell protein levels by immunohistochemistry (IHC) or mRNA levels by reverse transcriptase polymerase chain reaction (RT-PCR) or microarray analysis), in accordance with techniques and approaches disclosed herein and as would be apparent to one of ordinary skill in the art upon a review of the instant disclosure.

### VII. METHODS OF GENE EXPRESSION ANALYSIS

VII.A. Assay Formats

The genes identified herein in the study of melanoma can be used in a variety of nucleic acid detection assays to detect or quantitate the expression level of a gene or multiple genes in a given sample. For example, Northern blotting, nuclease protection, RT-PCR (e.g., quantitative RT-PCR (QRT-PCR)), and/or differential display methods can be used for detecting gene expression levels. In some embodiments, methods and assays of the presently disclosed subject matter are employed with array or chip hybridization-based methods for detecting the expression of a plurality of genes.

Any hybridization assay format can be used, including solution-based and solid support-based assay formats. Representative solid supports containing oligonucleotide probes for differentially expressed genes of the presently disclosed subject matter can be filters, polyvinyl chloride dishes, silicon, glass based chips, etc. Such wafers and hybridization methods are widely available and include, for example, those disclosed in PCT International Patent Application Publication WO 1995/11755. Any solid surface to which oligonucleotides can be bound, either directly or indirectly, either covalently or non-covalently, can be used. An exemplary solid support is a high-density array or DNA chip. These contain a particular oligonucleotide probe in a predetermined location on the array. Each predetermined location can contain more than one molecule of the probe, but in some embodiments each molecule within the predetermined location has an identical sequence. Such predetermined locations are termed features. There can be any number of features on a single solid support including, for example, about 2, 10, 100, 1000, 10,000, 100,000, or 400,000 of such features on a single solid support. The solid support, or the area within which the probes are attached, can be of any convenient size (for example, on the order of a square centimeter).

Oligonucleotide probe arrays for differential gene expression monitoring can be made and employed according to any techniques known in the art (see e.g., Lockhart et al., 1996; McGall et al., 1996). Such probe arrays can contain at least two or more oligonucleotides that are complementary to or hybridize to two or more of the genes described herein. Such arrays can also contain oligonucleotides that are complementary or hybridize to at least about 2, 3, 4, 5, 6, 7, 8, 9, 10, 15, 20, 25, 30, 50, 70, 100, or more of the nucleic acid sequences disclosed herein.

The genes that are assayed according to the presently disclosed subject matter are typically in the form of RNA (e.g., total RNA or mRNA) or reverse transcribed RNA. The genes can be cloned or not, and the genes can be amplified or not. In some embodiments, poly A⁺ RNA is employed as a source.

Probes based on the sequences of the genes described herein can be prepared by any commonly available method. Oligonucleotide probes for assaying the tissue or cell sample are in some embodiments of sufficient length to specifically hybridize only to appropriate complementary genes or transcripts. Typically, the oligonucleotide probes are at least 10, 12, 14, 16, 18, 20, or 25 nucleotides in length. In some embodiments, longer probes of at least 30, 40, 50, or 60 nucleotides are employed.

As used herein, oligonucleotide sequences that are complementary to one or more of the genes described herein are oligonucleotides that are capable of hybridizing under stringent conditions to at least part of the nucleotide sequence of said genes. Such hybridizable oligonucleotides will typically exhibit in some embodiments at least about 75% sequence identity, in some embodiments about 80% sequence identity, in some embodiments about 85% sequence identity, in some embodiments about 90% sequence identity, in some embodiments about 91% sequence identity, in some embodiments about 92% sequence identity, in some embodiments about 93% sequence identity, in some embodiments about 94% sequence identity, in some embodiments about 95% sequence identity, and in some embodiments greater than 95% sequence identity (e.g., 96%, 97%, 98%, 99%, or 100% sequence identity) at the nucleotide level to the nucleic acid sequences disclosed herein.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals can also be produced by intrinsic fluorescence of the array components themselves. A single background signal can be calculated for the entire array, or a different background signal can be calculated for each target nucleic acid. In some embodiments, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background can be calculated as the average hybridization signal intensity produced by hybridization to probes that are not complementary to any sequence found in the sample (e.g., probes directed to nucleic acids of the opposite sense or to genes not found in the sample such as bacterial genes where the sample is mammalian nucleic acids). Background can also be calculated as the average signal intensity produced by regions of the array that lack probes.

Assays and methods of the presently disclosed subject matter can utilize available formats to simultaneously screen in some embodiments at least about 10, in some embodiments at least about 50, in some embodiments at least about 100, in some embodiments at least about 1000, in some embodiments at least about 10,000, and in some embodiments at least about 40,000 or more different nucleic acid hybridizations.

The terms “mismatch control” and “mismatch probe” refer to a probe comprising a sequence that is deliberately selected not to be perfectly complementary to a particular target sequence. For each mismatch (MM) control in a high-density array there typically exists a corresponding perfect match (PM) probe that is perfectly complementary to the same particular target sequence. The mismatch can comprise one or more bases.

While the mismatch(s) can be located anywhere in the mismatch probe, terminal mismatches are less desirable as a terminal mismatch is less likely to prevent hybridization of the target sequence. In some embodiments, the mismatch is located at or near the center of the probe such that the mismatch is most likely to destabilize the duplex with the target sequence under the test hybridization conditions.

The phrase “perfect match probe” refers to a probe that has a sequence that is perfectly complementary to a particular target sequence. The test probe is typically perfectly complementary to a portion (subsequence) of the target sequence. The perfect match (PM) probe can be a “test probe”, a “normalization control” probe, an expression level control probe, or the like. A perfect match control or perfect match probe is, however, distinguished from a “mismatch control” or “mismatch probe”.

As used herein, a “probe” is defined as a nucleic acid that is capable of binding to a target nucleic acid of complementary sequence through one or more types of chemical bonds, usually through complementary base pairing, usually through hydrogen bond formation. As used herein, a probe can include natural (i.e., A, G, U, C, or T) or modified bases (7-deazaguanosine, inosine, etc.). In addition, the bases in probes can be joined by a linkage other than a phosphodiester bond, so long as it does not interfere with hybridization. Thus, probes can be peptide nucleic acids in which the constituent bases are joined by peptide bonds rather than phosphodiester linkages.

VII.B. Probe Design

Upon review of the present disclosure, one of skill in the art will appreciate that an enormous number of array designs are suitable for the practice of the presently disclosed subject matter. The high-density array typically includes a number of probes that specifically hybridize to the sequences of interest. See PCT International Patent Application Publication WO 1999/32660, incorporated herein be reference in its entirety, for methods of producing probes for a given gene or genes. In addition, in some embodiments, the array includes one or more control probes.

High-density array chips of the presently disclosed subject matter include in some embodiments “test probes”. Test probes can be oligonucleotides that in some embodiments range from about 5 to about 500 or about 5 to about 50 nucleotides, in some embodiments from about 10 to about 40 nucleotides, and in some embodiments from about 15 to about 40 nucleotides in length. In some embodiments, the probes are about 20 to 25 nucleotides in length. In some embodiments, test probes are double or single strand DNA sequences. DNA sequences are isolated or cloned from natural sources and/or amplified from natural sources using natural nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high-density array can contain a number of control probes. The control probes fall into three categories referred to herein as (1) normalization controls; (2) expression level controls; and (3) mismatch controls.

Normalization controls are oligonucleotide or other nucleic acid probes that are complementary to labeled reference oligonucleotides or other nucleic acid sequences that are added to the nucleic acid sample. The signals obtained from the normalization controls after hybridization provide a control for variations in hybridization conditions, label intensity, “reading” efficiency and other factors that can cause the signal of a perfect hybridization to vary between arrays. In some embodiments, signals (e.g., fluorescence intensity) read from some or all other probes in the array are divided by the signal (e.g., fluorescence intensity) from the control probes, thereby normalizing the measurements.

Virtually any probe can serve as a normalization control. However, it is recognized that hybridization efficiency varies with base composition and probe length. Exemplary normalization probes can be selected to reflect the average length of the other probes present in the array; however, they can be selected to cover a range of lengths. The normalization control(s) can also be selected to reflect the (average) base composition of the other probes in the array; however, in some embodiments, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typical expression level control probes have sequences complementary to subsequences of constitutively expressed “housekeeping genes” including, but not limited to, the β-actin gene, the transferrin receptor gene, the GAPDH gene, and the like.

Mismatch controls can also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). In some embodiments, mismatch probes contain one or more central mismatches. Thus, for example, where a probe is a 20-mer, a corresponding mismatch probe will have the identical sequence except for a single base mismatch (e.g., substituting a G, a C, or a T for an A) at any of positions 6 through 14 (the central mismatch).

Mismatch probes thus provide a control for non-specific binding or cross hybridization to a nucleic acid in the sample other than the target to which the probe is directed. Mismatch probes also indicate whether a hybridization is specific or not. For example, if the target is present the perfect match probes should be consistently brighter than the mismatch probes. In addition, if all central mismatches are present, the mismatch probes can be used to detect a mutation. The difference in intensity between the perfect match and the mismatch probe (IBM)-I(MM)) provides a good measure of the concentration of the hybridized material.

VII.C. Nucleic Acid Samples

A biological sample that can be analyzed in accordance with the presently disclosed subject matter comprises in some embodiments a nucleic acid. The terms “nucleic acid”, “nucleic acids”, and “nucleic acid molecules” each refer in some embodiments to deoxyribonucleotides, ribonucleotides, and polymers and folded structures thereof in either single- or double-stranded form. Nucleic acids can be derived from any source, including any organism. Deoxyribonucleic acids can comprise genomic DNA, cDNA derived from ribonucleic acid, DNA from an organelle (e.g., mitochondrial DNA or chloroplast DNA), or combinations thereof. Ribonucleic acids can comprise genomic RNA (e.g., viral genomic RNA), messenger RNA (mRNA), ribosomal RNA (rRNA), transfer RNA (tRNA), or combinations thereof.

VII.C.1. Isolation of Nucleic Acid Samples

Nucleic acid samples used in the methods and assays of the presently disclosed subject matter can be prepared by any available method or process. Methods of isolating total mRNA are also known to those of skill in the art. For example, methods of isolation and purification of nucleic acids are described in detail in Chapter 3 of Tijssen, 1993. Such samples include RNA samples, but also include cDNA synthesized from an mRNA sample isolated from a cell or tissue of interest. Such samples also include DNA amplified from the cDNA, an RNA transcribed from the amplified DNA, and combinations thereof. One of skill in the art would appreciate that it can be desirable to inhibit or destroy RNase present in homogenates before homogenates are used as a source of RNA.

The presently disclosed subject matter encompasses use of a sufficiently large biological sample to enable a comprehensive survey of low abundance nucleic acids in the sample. Thus, the sample can optionally be concentrated prior to isolation of nucleic acids. Several protocols for concentration have been developed that alternatively use slide supports (see Kohsaka & Carson, 1994; and Millar et al., 1995), filtration columns (see Bej et al., 1991), or immunomagnetic beads (see Albert et al., 1992; and Chiodi et al., 1992). Such approaches can significantly increase the sensitivity of subsequent detection methods.

As one example, SEPHADEX® matrix (Sigma of St. Louis, Mo., United States of America) is a matrix of diatomaceous earth and glass suspended in a solution of chaotropic agents and has been used to bind nucleic acid material. See Boom et al., 1990; and Buffone et al., 1991. After the nucleic acid is bound to the solid support material, impurities and inhibitors are removed by washing and centrifugation, and the nucleic acid is then eluted into a standard buffer. Target capture also allows the target sample to be concentrated into a minimal volume, facilitating the automation and reproducibility of subsequent analyses. See Lanciotti et al., 1992.

Methods for nucleic acid isolation can comprise simultaneous isolation of total nucleic acid, or separate and/or sequential isolation of individual nucleic acid types (e.g., genomic DNA, cDNA, organelle DNA, genomic RNA, mRNA, poly A⁺ RNA, rRNA, tRNA) followed by optional combination of multiple nucleic acid types into a single sample.

When RNA (e.g., mRNA) is selected for analysis, the disclosed methods allow for an assessment of gene expression in the tissue or cell type from which the RNA was isolated. RNA isolation methods are known to one of skill in the art. See Albert et al., 1992; Busch et al., 1992; Hamel et al., 1995; Herrewegh et al., 1995; Izraeli et al., 1991; McCaustland et al., 1991; Natarajan et al., 1994; Rupp et al., 1988; Tanaka et al., 1994; and Vankerckhoven et al., 1994.

Simple and semi-automated extraction methods can also be used for nucleic acid isolation, including for example, the SPLIT SECOND™ system (Boehringer Mannheim of Indianapolis, Ind., United States of America), the TRIZOL™ Reagent system (Life Technologies of Gaithersburg, Md., United States of America), and the FASTPREP™ system (Bio 101 of La Jolla, Calif., United States of America). See also Smith, 1998; and Paladichuk, 1999.

In some embodiments, nucleic acids that are used for subsequent amplification and labeling are analytically pure as determined by spectrophotometric measurements or by visual inspection following electrophoretic resolution. In some embodiments, the nucleic acid sample is free of contaminants such as polysaccharides, proteins, and inhibitors of enzyme reactions. When a biological sample comprises an RNA molecule that is intended for use in producing a probe, it is preferably free of DNase and RNase. Contaminants and inhibitors can be removed or substantially reduced using resins for DNA extraction (e.g., CHELEX™ 100 from BioRad Laboratories of Hercules, Calif., United States of America) or by standard phenol extraction and ethanol precipitation.

VII.C.2. Amplification of Nucleic Acid Samples

In some embodiments, a nucleic acid isolated from a biological sample is amplified prior to being used in the methods disclosed herein. In some embodiments, the nucleic acid is an RNA molecule, which is converted to a complementary DNA (cDNA) prior to amplification. Techniques for the isolation of RNA molecules and the production of cDNA molecules from the RNA molecules are known. See generally, Silhavy et al., 1984; Sambrook & Russell, 2001; Ausubel et al., 2002; and Ausubel et al., 2003). In some embodiments, the amplification of RNA molecules isolated from a biological sample is a quantitative amplification (e.g., by quantitative RT-PCR).

The terms “template nucleic acid” and “target nucleic acid” as used herein each refer to nucleic acids isolated from a biological sample as described herein above. The terms “template nucleic acid pool”, “template pool”, “target nucleic acid pool”, and “target pool” each refer to an amplified sample of “template nucleic acid”. Thus, a target pool comprises amplicons generated by performing an amplification reaction using the template nucleic acid. In some embodiments, a target pool is amplified using a random amplification procedure as described herein.

The term “target-specific primer” refers to a primer that hybridizes selectively and predictably to a target sequence, for example a subsequence of one of the six genes disclosed herein, in a target nucleic acid sample. A target-specific primer can be selected or synthesized to be complementary to known nucleotide sequences of target nucleic acids.

The term “random primer” refers to a primer having an arbitrary sequence. The nucleotide sequence of a random primer can be known, although such sequence is considered arbitrary in that it is not specifically designed for complementarity to a nucleotide sequence of the presently disclosed subject matter. The term “random primer” encompasses selection of an arbitrary sequence having increased probability to be efficiently utilized in an amplification reaction. For example, the Random Oligonucleotide Construction Kit (ROCK) is a macro-based program that facilitates the generation and analysis of random oligonucleotide primers. See Strain & Chmielewski, 2001. Representative primers include but are not limited to random hexamers and rapid amplification of polymorphic DNA (RAPD)-type primers as described by Williams et al., 1990.

A random primer can also be degenerate or partially degenerate as described by Telenius et al., 1992. Briefly, degeneracy can be introduced by selection of alternate oligonucleotide sequences that can encode a same amino acid sequence.

In some embodiments, random primers can be prepared by shearing or digesting a portion of the template nucleic acid sample. Random primers so-constructed comprise a sample-specific set of random primers.

The term “heterologous primer” refers to a primer complementary to a sequence that has been introduced into the template nucleic acid pool. For example, a primer that is complementary to a linker or adaptor, as described below, is a heterologous primer. Representative heterologous primers can optionally include a poly(dT) primer, a poly(T) primer, or as appropriate, a poly(dA) or poly(A) primer.

The term “primer” as used herein refers to a contiguous sequence comprising in some embodiments about 6 or more nucleotides, in some embodiments about 10-20 nucleotides (e.g., 15-mer), and in some embodiments about 20-30 nucleotides (e.g., a 22-mer). Primers used to perform the methods of the presently disclosed subject matter encompass oligonucleotides of sufficient length and appropriate sequence so as to provide initiation of polymerization on a nucleic acid molecule.

U.S. Pat. No. 6,066,457 to Hampson et al. describes a method for substantially uniform amplification of a collection of single stranded nucleic acid molecules such as RNA. Briefly, the nucleic acid starting material is anchored and processed to produce a mixture of directional shorter random size DNA molecules suitable for amplification of the sample.

In accordance with the methods of the presently disclosed subject matter, any PCR technique or related technique can be employed to perform the step of amplifying the nucleic acid sample. In addition, such methods can be optimized for amplification of a particular subset of nucleic acid (e.g., genomic DNA versus RNA), and representative optimization criteria and related guidance can be found in the art. See Cha & Thilly, 1993; Linz et al., 1990; Robertson & Walsh-Weller, 1998; Roux, 1995; Williams, 1989; and McPherson et al., 1995.

VII.C.3. Labeling of Nucleic Acid Samples

Optionally, a nucleic acid sample (e.g., a quantitatively amplified RNA sample) further comprises a detectable label. In some embodiments of the presently disclosed subject matter, the amplified nucleic acids can be labeled prior to hybridization to an array. Alternatively, randomly amplified nucleic acids are hybridized with a set of probes, without prior labeling of the amplified nucleic acids. For example, an unlabeled nucleic acid in the biological sample can be detected by hybridization to a labeled probe. In some embodiments, both the randomly amplified nucleic acids and the one or more pathogen-specific probes include a label, wherein the proximity of the labels following hybridization enables detection. An exemplary procedure using nucleic acids labeled with chromophores and fluorophores to generate detectable photonic structures is described in U.S. Pat. No. 6,162,603 to Heller.

In accordance with the methods of the presently disclosed subject matter, the amplified nucleic acids and/or probes/probe sets can be labeled using any detectable label. It will be understood to one of skill in the art that any suitable method for labeling can be used, and no particular detectable label or technique for labeling should be construed as a limitation of the disclosed methods.

Direct labeling techniques include incorporation of radioisotopic or fluorescent nucleotide analogues into nucleic acids by enzymatic synthesis in the presence of labeled nucleotides or labeled PCR primers. A radio-isotopic label can be detected using autoradiography or phosphorimaging. A fluorescent label can be detected directly using emission and absorbance spectra that are appropriate for the particular label used. Any detectable fluorescent dye can be used, including but not limited to FITC (fluorescein isothiocyanate), FLUOR X™, ALEXA FLUOR® 488, OREGON GREEN® 488, 6-JOE (6-carboxy-4′,5′-dichloro-2′,7′-dimethoxyfluorescein, succinimidyl ester), ALEXA FLUOR® 532, Cy3, ALEXA FLUOR® 546, TMR (tetramethylrhodamine), ALEXA FLUOR® 568, ROX (X-rhodamine), ALEXA FLUOR® 594, TEXAS RED®, BODIPY® 630/650, and Cy5 (available from Amersham Pharmacia Biotech of Piscataway, N.J., United States of America or from Molecular Probes Inc. of Eugene, Oreg., United States of America). Fluorescent tags also include sulfonated cyanine dyes (available from Li-Cor, Inc. of Lincoln, Nebr., United States of America) that can be detected using infrared imaging. Methods for direct labeling of a heterogeneous nucleic acid sample are known in the art and representative protocols can be found in, for example, DeRisi et al., 1996; Sapolsky & Lipshutz, 1996; Schena et al., 1995; Schena et al., 1996; Shalon et al., 1996; Shoemaker et al., 1996; and Wang et al., 1998.

In some embodiments, nucleic acid molecules isolated from different cell types (e.g., primary versus metastatic melanoma) are labeled with different detectable markers, allowing the nucleic acids to analyzed simultaneously on an array. For example, a first RNA sample can be reverse transcribed into cDNAs labeled with cyanine 3 (a green dye fluorophore; Cy3) while a second RNA sample to which the first RNA sample is to be compared can be labeled with cyanine 5 (a red dye fluorophore; Cy5).

The quality of probe or nucleic acid sample labeling can be approximated by determining the specific activity of label incorporation. For example, in the case of a fluorescent label, the specific activity of incorporation can be determined by the absorbance at 260 nm and 550 nm (for Cy3) or 650 nm (for Cy5) using published extinction coefficients. See Randolph & Waggoner, 1995. Very high label incorporation (specific activities of >1 fluorescent molecule/20 nucleotides) can result in a decreased hybridization signal compared with probe with lower label incorporation. Very low specific activity (<1 fluorescent molecule/100 nucleotides) can give unacceptably low hybridization signals. See Worley et al., 2000. Thus, it will be understood to one of skill in the art that labeling methods can be optimized for performance in microarray hybridization assay, and that optimal labeling can be unique to each label type.

VII.D. Forming High-Density Arrays

In some embodiments of the presently disclosed subject matter, probes or probe sets are immobilized on a solid support such that a position on the support identifies a particular probe or probe set. In the case of a probe set, constituent probes of the probe set can be combined prior to placement on the solid support or by serial placement of constituent probes at a same position on the solid support.

A microarray can be assembled using any suitable method known to one of skill in the art, and any one microarray configuration or method of construction is not considered to be a limitation of the presently disclosed subject matter. Representative microarray formats that can be used in accordance with the methods of the presently disclosed subject matter are described herein below and include, but are not limited to light-directed chemical coupling, and mechanically directed coupling. See U.S. Pat. No. 5,143,854 to Pirrung et al.; U.S. Pat. No. 5,800,992 to Fodor et al.; and U.S. Pat. No. 5,837,832 to Chee et al.

VII.E. Hybridization

VII.E.1. General Considerations

The terms “specifically hybridizes” and “selectively hybridizes” each refer to binding, duplexing, or hybridizing of a molecule only to a particular nucleotide sequence under stringent conditions when that sequence is present in a complex nucleic acid mixture (e.g., total cellular DNA or RNA).

The phrase “substantially hybridizes” refers to complementary hybridization between a probe nucleic acid molecule and a substantially identical target nucleic acid molecule as defined herein. Substantial hybridization is generally permitted by reducing the stringency of the hybridization conditions using art-recognized techniques.

“Stringent hybridization conditions” and “stringent hybridization wash conditions” in the context of nucleic acid hybridization experiments are both sequence- and environment-dependent. Longer sequences hybridize specifically at higher temperatures. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. Typically, under “stringent conditions” a probe hybridizes specifically to its target sequence, but to no other sequences.

An extensive guide to the hybridization of nucleic acids is found in Tijssen, 1993. In general, a signal to noise ratio of 2-fold (or higher) than that observed for a negative control probe in a same hybridization assay indicates detection of specific or substantial hybridization.

VII.E.2. Hybridization on a Solid Support

In some embodiments of the presently disclosed subject matter, an amplified and/or labeled nucleic acid sample is hybridized to specific probes or probe sets that are immobilized on a continuous solid support comprising a plurality of identifying positions. Representative formats of such solid supports are described herein.

The following are examples of hybridization and wash conditions that can be used to clone homologous nucleotide sequences that are substantially identical to reference nucleotide sequences of the presently disclosed subject matter: a probe nucleotide sequence hybridizes in one example to a target nucleotide sequence in 7% sodium dodecyl sulfate (SDS), 0.5M NaPO₄, 1 mm ethylene diamine tetraacetic acid (EDTA), 1% BSA at 50° C. followed by washing in 2×SSC, 0.1% SDS at 50° C.; in another example, a probe and target sequence hybridize in 7% SDS, 0.5 M NaPO₄, 1 mm EDTA, 1% BSA at 50° C. followed by washing in 1×SSC, 0.1% SDS at 50° C.; in another example, a probe and target sequence hybridize in 7% SDS, 0.5 M NaPO₄, 1 mm EDTA, 1% BSA at 50° C. followed by washing in 0.5×SSC, 0.1% SDS at 50° C.; in another example, a probe and target sequence hybridize in 7% SDS, 0.5 M NaPO₄, 1 mm EDTA, 1% BSA at 50° C. followed by washing in 0.1×SSC, 0.1% SDS at 50° C.; in yet another example, a probe and target sequence hybridize in 7% SDS, 0.5 M NaPO₄, 1 mm EDTA, 1% BSA at 50° C. followed by washing in 0.1×SSC, 0.1% SDS at 65° C. In some embodiments, hybridization conditions comprise hybridization in a roller tube for at least 12 hours at 42° C. In each of the above conditions, the sodium phosphate hybridization buffer can be replaced by a hybridization buffer comprising 6×SSC (or 6×SSPE), 5×Denhardt's reagent, 0.5% SDS, and 100 g/ml carrier DNA, including 0-50% formamide, with hybridization and wash temperatures chosen based upon the desired stringency. Other hybridization and wash conditions are known to those of skill in the art (see also Sambrook & Russell, 2001; Ausubel et al., 2002; and Ausubel et al., 2003; each of which is incorporated herein in its entirety). As is known in the art, the addition of formamide in the hybridization solution reduces the Tₘ by about 0.4° C. Thus, high stringency conditions include the use of any of the above solutions and 0% formamide at 65° C., or any of the above solutions plus 50% formamide at 42° C.

For some high-density glass-based microarray experiments, hybridization at 65° C. is too stringent for typical use, at least in part because the presence of fluorescent labels destabilizes the nucleic acid duplexes. See Randolph & Waggoner, 1995. Alternatively, hybridization can be performed in a formamide-based hybridization buffer as described in Piétu et al., 1996.

A microarray format can be selected for use based on its suitability for electrochemical-enhanced hybridization. Provision of an electric current to the microarray, or to one or more discrete positions on the microarray facilitates localization of a target nucleic acid sample near probes immobilized on the microarray surface. Concentration of target nucleic acid near arrayed probe accelerates hybridization of a nucleic acid of the sample to a probe. Further, electronic stringency control allows the removal of unbound and nonspecifically bound DNA after hybridization. See U.S. Pat. No. 6,017,696 to Heller and U.S. Pat. No. 6,245,508 to Heller & Sosnowski.

II.E.3. Hybridization in Solution

In some embodiments of the presently disclosed subject matter, an amplified and/or labeled nucleic acid sample is hybridized to one or more probes in solution. Representative stringent hybridization conditions for complementary nucleic acids having more than about 100 complementary residues are overnight hybridization in 50% formamide with 1 mg of heparin at 42° C. An example of highly stringent wash conditions is 15 minutes in 0.1×SSC, 5 M NaCl at 65° C. An example of stringent wash conditions is 15 minutes in 0.2×SSC buffer at 65° C. (see Sambrook and Russell, 2001, for a description of SSC buffer). A high stringency wash can be preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4-6×SSC at 40° C. Stringent conditions can also be achieved with the addition of destabilizing agents such as formamide.

For short probes (e.g., about 10 to 50 nucleotides), stringent conditions typically involve salt concentrations of less than about 1M Na⁺ ion, typically about 0.01 M to 1 M Na⁺ ion concentration (or other salts) at pH 7.0-8.3, and the temperature is typically at least about 30° C.

Optionally, nucleic acid duplexes or hybrids can be captured from the solution for subsequent analysis, including detection assays. For example, in a simple assay, a single pathogen-specific probe set is hybridized to an amplified and labeled RNA sample derived from a target nucleic acid sample. Following hybridization, an antibody that recognizes DNA:RNA hybrids is used to precipitate the hybrids for subsequent analysis. The presence of the pathogen is determined by detection of the label in the precipitate.

Alternate capture techniques can be used as will be understood to one of skill in the art, for example, purification by a metal affinity column when using probes comprising a histidine tag. As another example, the hybridized sample can be hydrolyzed by alkaline treatment wherein the double-stranded hybrids are protected while non-hybridizing single-stranded template and excess probe are hydrolyzed. The hybrids are then collected using any nucleic acid purification technique for further analysis.

To assess the expression of multiple genes and/or samples from multiple different sources simultaneously, probes or probe sets can be distinguished by differential labeling of probes or probe sets. Alternatively, probes or probe sets can be spatially separated in different hybridization vessels.

In some embodiments, a probe or probe set having a unique label is prepared for each gene or source to be detected. For example, a first probe or probe set can be labeled with a first fluorescent label, and a second probe or probe set can be labeled with a second fluorescent label. Multi-labeling experiments should consider label characteristics and detection techniques to optimize detection of each label. Representative first and second fluorescent labels are Cy3 and Cy5 (Amersham Pharmacia Biotech of Piscataway, N.J. United States of America), which can be analyzed with good contrast and minimal signal leakage.

A unique label for each probe or probe set can further comprise a labeled microsphere to which a probe or probe set is attached. A representative system is LabMAP (Luminex Corporation of Austin, Tex., United States of America). Briefly, LabMAP (Laboratory Multiple Analyte Profiling) technology involves performing molecular reactions, including hybridization reactions, on the surface of color-coded microscopic beads called microspheres. When used in accordance with the methods of the presently disclosed subject matter, an individual pathogen-specific probe or probe set is attached to beads having a single color-code such that they can be identified throughout the assay. Successful hybridization is measured using a detectable label of the amplified nucleic acid sample, wherein the detectable label can be distinguished from each color-code used to identify individual microspheres. Following hybridization of the randomly amplified, labeled nucleic acid sample with a set of microspheres comprising pathogen-specific probe sets, the hybridization mixture is analyzed to detect the signal of the color-code as well as the label of a sample nucleic acid bound to the microsphere. See Vignali 2000; Smith et al., 1998; and PCT International Patent Application Publication Nos. WO 2001/13120; WO 2001/14589; WO 1999/19515; WO 1999/32660; and WO 1997/14028.

VII.F. Detection

Methods for detecting hybridization are typically selected according to the label employed.

In the case of a radioactive label (e.g., ³²P-dNTP) detection can be accomplished by autoradiography or by using a phosphorimager as is known to one of skill in the art. In some embodiments, a detection method can be automated and is adapted for simultaneous detection of numerous samples.

Common research equipment has been developed to perform high-throughput fluorescence detecting, including instruments from GSI Lumonics (Watertown, Mass., United States of America), Amersham Pharmacia Biotech/Molecular Dynamics (Sunnyvale, Calif., United States of America), Applied Precision Inc. (Issauah, Wash., United States of America), Genomic Solutions Inc. (Ann Arbor, Mich., United States of America), Genetic MicroSystems Inc. (Woburn, Mass., United States of America), Axon (Foster City, Calif., United States of America), Hewlett Packard (Palo Alto, Calif., United States of America), and Virtek (Woburn, Mass., United States of America). Most of the commercial systems use some form of scanning technology with photomultiplier tube detection. Criteria for consideration when analyzing fluorescent samples are summarized by Alexay et al., 1996.

In some embodiments, a nucleic acid sample or probe is labeled with far infrared, near infrared, or infrared fluorescent dyes. Following hybridization, the mixture of nucleic acids and probes is scanned photoelectrically with a laser diode and a sensor, wherein the laser scans with scanning light at a wavelength within the absorbance spectrum of the fluorescent label, and light is sensed at the emission wavelength of the label. See U.S. Pat. No. 6,086,737 to Patonay et al.; U.S. Pat. No. 5,571,388 to Patonay et al.; U.S. Pat. No. 5,346,603 to Middendorf & Brumbaugh; U.S. Pat. No. 5,534,125 to Middendorf et al.; U.S. Pat. No. 5,360,523 to Middendorf et al.; U.S. Pat. No. 5,230,781 to Middendorf & Patonay; U.S. Pat. No. 5,207,880 to Middendorf & Brumbaugh; and U.S. Pat. No. 4,729,947 to Middendorf & Brumbaugh. An ODYSSEY™ infrared imaging system (Li-Cor, Inc. of Lincoln, Nebr., United States of America) can be used for data collection and analysis. If an epitope label has been used, a protein or compound that binds the epitope can be used to detect the epitope. For example, an enzyme-linked protein can be subsequently detected by development of a colorimetric or luminescent reaction product that is measurable using a spectrophotometer or luminometer, respectively.

In some embodiments, INVADER® technology (Third Wave Technologies of Madison, Wis., United States of America) is used to detect target nucleic acid/probe complexes. Briefly, a nucleic acid cleavage site (such as that recognized by a variety of enzymes having 5′ nuclease activity) is created on a target sequence, and the target sequence is cleaved in a site-specific manner, thereby indicating the presence of specific nucleic acid sequences or specific variations thereof. See U.S. Pat. No. 5,846,717 to Brow et al.; U.S. Pat. No. 5,985,557 to Prudent et al.; U.S. Pat. No. 5,994,069 to Hall et al.; U.S. Pat. No. 6,001,567 to Brow et al.; and U.S. Pat. No. 6,090,543 to Prudent et al.

In some embodiments, target nucleic acid/probe complexes are detected using an amplifying molecule, for example a poly-dA oligonucleotide as described by Lisle et al., 2001. Briefly, a tethered probe is employed against a target nucleic acid having a complementary nucleotide sequence. A target nucleic acid having a poly-dT sequence, which can be added to any nucleic acid sequence using methods known to one of skill in the art, hybridizes with an amplifying molecule comprising a poly-dA oligonucleotide. Short oligo-dT₄₀ signaling moieties are labeled with any suitable label (e.g., fluorescent, chemiluminescent, radioisotopic labels). The short oligo-dT₄₀ signaling moieties are subsequently hybridized along the molecule, and the label is detected.

The presently disclosed subject matter also envisions use of electrochemical technology for detecting a nucleic acid hybrid according to the disclosed method. In this case, the detection method relies on the inherent properties of DNA, and thus a detectable label on the target sample or the probe/probe set is not required. In some embodiments, probe-coupled electrodes are multiplexed to simultaneously detect multiple genes using any suitable microarray or multiplexed liquid hybridization format. To enable detection, gene-specific and control probes are synthesized with substitution of the non-physiological nucleic acid base inosine for guanine, and subsequently coupled to an electrode. Following hybridization of a nucleic acid sample with probe-coupled electrodes, a soluble redox-active mediator (e.g., ruthenium 2,2′-bipyridine) is added, and a potential is applied to the sample. In the absence of guanine, each mediator is oxidized only once. However, when a guanine-containing nucleic acid is present, by virtue of hybridization of a sample nucleic acid molecule to the probe, a catalytic cycle is created that results in the oxidation of guanine and a measurable current enhancement. See U.S. Pat. No. 6,127,127 to Eckhardt et al.; U.S. Pat. No. 5,968,745 to Thorp et al.; and U.S. Pat. No. 5,871,918 to Thorp et al.

Surface plasmon resonance spectroscopy can also be used to detect hybridization. See e.g., Heaton et al., 2001; Nelson et al., 2001; and Guedon et al., 2000.

VII.G. Data Analysis

Databases and software designed for use with microarrays is discussed in U.S. Pat. No. 6,229,911 to Balaban & Aggarwal, which describes a computer-implemented method for managing information, stored as indexed tables, collected from small or large numbers of microarrays, and in U.S. Pat. No. 6,185,561 to Balaban & Khurgin, which describes a computer-based method with data mining capability for collecting gene expression level data, adding additional attributes and reformatting the data to produce answers to various queries. U.S. Pat. No. 5,974,164 to Chee describes a software-based method for identifying mutations in a nucleic acid sequence based on differences in probe fluorescence intensities between wild type and mutant sequences that hybridize to reference sequences.

Analysis of microarray data can also be performed using the method disclosed in Tusher et al., 2001, which describes the Significance Analysis of Microarrays (SAM) method for determining significant differences in gene expression among two or more samples.

### VIII. ARRAYS, KITS, AND COMPOSITIONS FOR USE IN THE PRESENTLY DISCLOSED METHODS

The presently disclosed subject matter also provides arrays, kits, and compositions that can be employed in the practice of the methods disclosed herein.

As is known to one of ordinary skill in the art, gene expression levels can be assayed either at the level of RNA or at the level of protein. As such, in some embodiments RNA is extracted from the biological sample and analyzed by techniques that include, but are not limited to PCR analysis (in some embodiments, quantitative RT-PCR) and/or array analysis. In each case, one of ordinary skill in the art would be aware of techniques that can be employed to determine the expression level of a gene product in the biological sample.

With respect to PCR analyses, the sequences of nucleic acids that correspond to exemplary LKB1, YES, and/or CD24 gene products are present within the GENBANK® database (a subset of which are also provided in the Sequence Listing), and oligonucleotide primers can be designed for the purpose of determining expression levels.

Alternatively, arrays can be produced that include single-stranded nucleic acids that can hybridize to LKB1, YES, and/or CD24 gene products. Exemplary, non-limiting methods that can be used to produce and screen arrays are described in Section VII hereinabove.

Therefore, in some embodiments the presently disclosed subject matter provides arrays comprising polynucleotides that are capable of hybridizing to at least two genes selected from among LKB1, YES, and/or CD24 or comprising specific peptide or polypeptide gene products of LKB1, YES, and/or CD24.

Alternatively or in addition, gene expression can be assayed by determining the levels at which polypeptides are present in melanoma tissue. This can also be done using arrays, and exemplary methods for producing peptide and/or polypeptide arrays attached to nitrocellulose-coated glass slides, alkanethiol-coated gold surfaces, poly-L-lysine-treated glass slides, aldehyde-treated glass slides, silane-modified glass slides, and nickel-treated glass slides, among others, have been reported.

In addition to the description above, U.S. Patent Application Publication No. 2011/0119776, incorporated herein by reference in its entirety, also provides information and methodology regarding gene expression profiles, particularly in the context of LKB1 expression and lung cancer.

In some embodiments the presently disclosed subject matter provides arrays that comprise peptides or polypeptides that are correspond to gene products from one or more (e.g., two or three) of LKB1, YES, and CD24. In these embodiments, arrays are produced from proteins isolated from melanoma tissue, and these arrays are then probed with molecules that specifically bind to the various gene products of interest, if present. Exemplary molecules that specifically bind to LKB1, YES, and CD24 gene products include antibodies (as well as fragments and derivatives thereof that include at least one Fab fragment). Antibodies can be commercially available, and/or antibodies that specifically bind to LKB1, YES, or CD24 gene products can be produced using routine techniques. Thus, in some embodiments, “binding molecules” refer to antibodies and antibody fragments and derivatives that include at least one Fab fragment.

Peptide and/or polypeptide arrays can be designed quantitatively such that the amount of each individual peptide or polypeptide is reflective of the amount of that individual peptide or polypeptide in the melanoma tissue.

Further, the arrays can be designed such that specific peptide or polypeptide gene products that correspond to one or more of the LKB1, YES, and CD24 genes can be localized (sometimes referred to as “spotted”) on the array such that the array is interrogatable with at least one antibody that specifically binds to one of the specific peptide or polypeptide gene products.

In some embodiments, gene expression at the level of protein is assayed without isolating the relevant peptides and/or polypeptides from the melanoma cells. For example, immunohistochemistry and/or immunocytochemistry can be employed, in which the expression levels of gene products that correspond to one or more of the LKB1, YES, and/or CD24 genes can be determined by incubating appropriate binding molecules to melanoma cells and/or tissue. In some embodiments, the melanoma cells and/or tissue is mounted in paraffin blocks before the immunohistochemistry and/or immunocytochemistry is performed.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### General Methods

Mouse Colony:

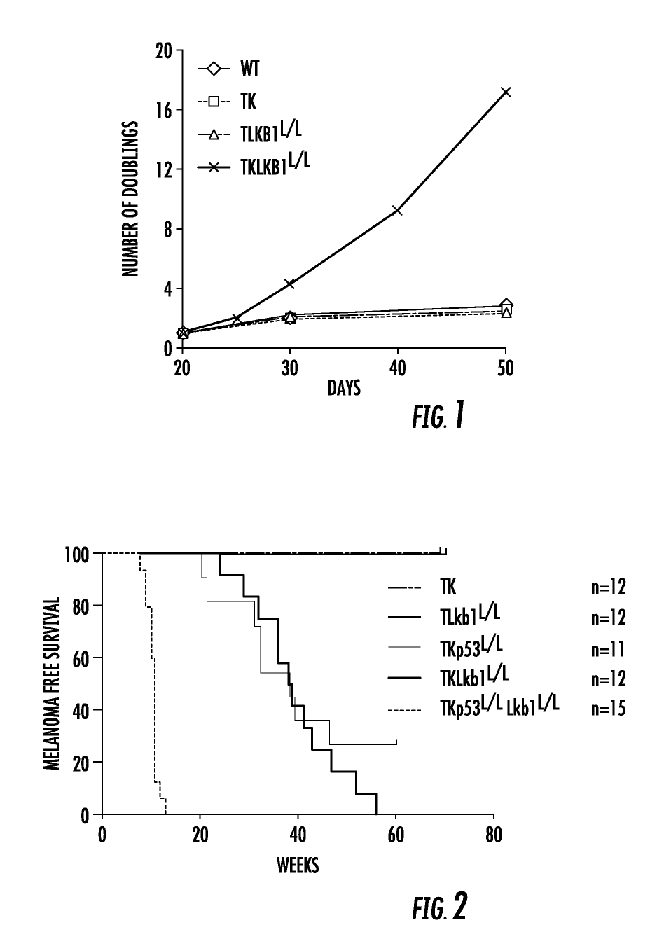

Mice were housed and treated in accordance with protocols approved by the institutional care and use committee for animal research at the University of North Carolina. Animals were generated and genotyped as previously described: Tyr-CRE-ER^{T2} (or “T”, (Bosenberg et al., 2006)), K-Ras^{L/L} (or “K”, (Johnson et al., 2001)), Lkb1^{L/L} (Bardeesy et al., 2002), p53^{L/L} (Jonkers et al., 2001), and Tyr-Ras Ink4a/Arf (Chin et al., 1997). All cohorts reported in FIGS. 1 and 2 (TK, TLkb1^{L/L}, Tp53^{L/L}, TLkb1^{L/L}p53^{L/L}, TKLkb1^{L/L}, TKp₅₃^{L/L}, TKLkb1^{L/L}p53^{L/L}) were newly generated and contemporaneously housed. Data from the TKp16^{L/L} and TKp53^{L/L}p16^{L/L} cohorts shown in Table 4, below, are a historical comparison from a prior study. See Monahan et al., 2010. All cohorts were N1 in C57BL/6, and, where possible, compared to littermate controls. To induce CRE recombinase in vivo, pups were treated on post-natal days 2, 3 and 4 with 4-hydroxy-tamoxifen (4-OHT, Sigma H7904, Sigma, St Louis, Mo., United States of America) at 25 mg/mL in dimethyl sulfoxide. In tumor survival cohorts, mice were monitored for tumors 3× per week, and sacrificed when tumors reached 1.3 cm in size or caused significant morbidity (e.g. weight loss, tumor ulceration). All sacrificed animals were analyzed for metastasis by gross autopsy. Hematoxylin and Eosin (H&E) staining of tumors after paraffin embedding and formalin fixation was performed, with analysis showing spindle shaped melanoma with variable degree of melanin. Melanocytic lineage was further confirmed by deriving cells lines from the primary tumors and metastases and staining for melanocytic markers. Kaplan-Meier analysis of melanoma-free survival was determined using GraphPad Prism software (GraphPad Software, La Jolla, San Diego, Calif., United States of America).

Cell Lines and Cell Culture:

Tumor cell lines were generated and maintained from mice of the indicated genotypes as previously described. See Sharpless et al., 2002. Primary melanocyte cultures were prepared as previously described (see Bennett et al., 1989; and Spanakis et al., 1992) and plated on collage-coated dishes. To induce CRE recombinase in vitro, primary melanocyte cultures were treated with or without 4-OHT at 20 days post-isolation for 48 hours.

Human A2058 cells and indicated murine melanoma cells were maintained at 37° C. in a 5% CO₂-humidified atmosphere on Dulbecco's modified Eagle's medium (DMEM) containing 10% fetal bovine serum (FBS) and 100 ng/mL each of penicillin and streptomycin. Dasatinib was purchased from LC Laboratories (D-3307; Woburn, Mass., United States of America) and dissolved in DMSO. For growth curve analysis, cells were counted with hemocytometer at indicated times.

Immunoprecipitation, Immunoblotting and Immunofluorescence:

Cell lysates were prepared in RIPA buffer with protease inhibitors (Roche, Indianapolis, Ind., United States of America) and phosphatase inhibitors (Calbiochem, EMD Chemicals Inc, Darmstadt, Germany). For immunoprecipitation, cell lysates were precleared with Protein A agarose beads for 1 hour, incubated with indicated antibody overnight at 4° C., mixed with Protein A agarose beads, incubated for 3 hours, and then washed with lysis buffer five times. The immunoprecipitates were then subjected to immunoblotting.

For immunoblotting, standard western blot procedures were performed after resolution on polyacrylamide gels. Antibodies used were 13-actin (C-1, Santa Cruz Biotechnology, Inc., Santa Cruz, Calif., United States of America), LKB1 (D6005, Cell Signaling Technology, Beverly, Mass., United States of America), Src (32G6, Cell Signaling Technology, Beverly, Mass., United States of America), Fyn (FYN3, Santa Cruz Biotechnology, Inc., Santa Cruz, Calif., United States of America), Yes (H-95, Santa Cruz Biotechnology, Santa Cruz, Calif., United States of America), p-SFK (100F9, Cell Signaling Technology, Beverly, Mass., United States of America), IRDye 680 Donkey anti-Rabbit IgG (926-32223, LiCor Biosciences, Lincoln, Nebr., United States of America), IRDye 8000W Donkey anti-Goat IgG (926-32214, LiCor Biosciences, Lincoln, Nebr., United States of America). Band intensity was quantified using a LiCor ODYSSEY® Infrared Imaging System (LiCor Biosciences, Lincoln, Nebr., United States of America).

For immunofluorescence, cells were grown on coverslips. After fixation in 4% paraformaldehyde, cells were permeabilized in 0.1% Triton X-100, blocked in 10% normal goat serum and incubated with indicated primary antibody for 1 hour. Cells were washed three times and then incubated with an Alexa Fluor 488-conjugated secondary anti-rabbit antibody for 45 minutes.

Cell Migration and Invasion Assays:

The in vitro scratch (wound healing) assay was performed as described previously. See Carretero et al., 2010. Briefly, a 1 mm wide scratch was made on a confluent monolayer, and cells were then allowed to grow under standard conditions for 12 hours. The migrated distance was quantified using Image J™ software. “Close Index” was determined as 1-f, where f is calculated as the remaining gap area divided by the starting scratched area. Cell invasion was measured using matrigel invasion assay using invasion chambers obtained from BD Biosciences (San Jose, Calif., United States of America), with assays performed according to the manufacturer's instructions. Cells of the indicated genotypes (2.5×10⁴) were added to the upper chamber in 500 uL of serum-free medium, and the lower chamber was filled with 750 uL of medium containing 10% fetal bovine serum (FBS) as an attractant. After 24 hours of incubation, cells on the underside of the filter were fixed, stained and counted. For dasatinib treatment, 30 nM dasatinib was added to both upper and lower chambers 6 hours after cells were seeded, to allow cell attachment.

Src 8-Plex Analysis:

Quantification of Src family kinase activities were assayed using SRC Family Kinase 8-Plex (Millipore Corporation, Billerica, Mass., United States of America). Assays were performed according to the manufacturer's specifications and analyzed with a LUMINEX™ 200 platform (Luminex Corporation, Austin, Tex., United States of America). Briefly, 20 mg of protein per sample was incubated with LUMINEX™ beads conjugated with SFK (Src family kinases) specific antibody. Following the incubation, the beads were washed and incubated with biotinylated antibody targeting tyrosine 419 on an active loop. The bead conjugates were then washed and incubated with phycoerythrin. Mean fluorescence intensity (MFI) from duplicate samples was averaged with background correction from duplicate samples. Statistical significance was calculated using a two-sided Student's exact t-test.

Flow Cytometric Analysis and Fluorescence-Activated Cell Sorting (FACS):

Cells were labeled with indicated antibodies, washed, resuspended and filtered through a 40-pm cell strainer. Data were recorded with a CyAn ADP flow cytometer (DAKO, Beckman Coulter, Brea, Calif., United States of America) and analyzed by FlowJo™ software (TreeStar, Inc., Ashland, Oregan, United States of America). Antibodies used were Anti-Human APC-CD24 (eBioscience, Inc., San Diego, Calif., United States of America), Anti-Mouse FITC-0D24 (eBioscience, Inc., San Diego, Calif., United States of America), Anti-Mouse PE-Cy5-CD24 (eBioscience, Inc., San Diego, Calif., United States of America), and Anti-Human FITC-CD44 (BD Biosciences, San Jose, Calif., United States of America). For colony-forming cell (CFC) assay, single cells were FACS-sorted into individual wells of 96-well plates. Colony-forming cells were counted after culturing the cells for three weeks.

Quantitative RT-PCR:

Total RNA was purified by using RNeasy Mini Kit (Qiagen, Valencia, Calif., United States of America) and SUPERSCRIPT® Synthesis System for RT-PCR (Invitrogen, Carlsbad, Calif., United States of America) was used to synthesize first-strand cDNA from total RNA. RT-PCR reactions were prepared in triplicate for each sample and run on 7900HT Fast Real-Time PCR System (Applied Biosystems, Foster City, Calif., United States of America). Taqman probe for human CD24 was purchased from Applied Biosystems (Foster City, Calif., United States of America). 18S (Applied Biosystems, Foster City, Calif., United States of America) was used as a reference for all reactions. Relative mRNA expression was determined by DDCt method.

Xenograft Experiments:

Five to six week-old female nu/nu mice were maintained under pathogen-free conditions. Cells were sorted by FACS and 5,000 sorted cells were injected subcutaneously on the dorsal side of the ears as previously reported. See Rozenberg et al., 2010. After three weeks, animals were sacrificed and tumor sizes were measured using calipers. Tumor volume was calculated as (length×width²)/2. A two-sided Student's two-tailed t-test was applied for statistical analyses.

Short Hairpin RNA Constructs, Lentiviral Infection, and Small Interfering RNA Transfection:

Short hairpin RNA (shRNA) constructs used for knocking down LKB1 expression in A2058 cells were described previously. See Carretero et al., 2010. For suppression of Src, Fyn, and Yes expression, cells were transfected with the appropriate antisense oligonucleotides using Lipofectamine RNA1MAX (Invitrogen, Carlsbad, Calif., United States of America). siRNAs used were Src siRNA (sc-29228, Santa Cruz), Fyn siRNA (sc-29321, Santa Cruz), Yes siRNA (sc-29860, Santa Cruz), and scrambled control siRNA (sc-37007, Santa Cruz), all from Santa Cruz Biotechnology, Inc. (Santa Cruz, Calif., United States of America).

### Example 2

### Lkb1 Restrains Melanocytic Hyperproliferation Induced by K-Ras Activation

To examine the role of Lkb1 in melanocyted growth and transformation, an established 4-hydroxytamoxifen (4-OHT)-inducible melanocyte-specific CRE allele (Tyr-CRE-ER^{T2} (abbreviated “T”, see Bosenberg et al., 2006)) and three conditional alleles: Lox-Stop-Lox-(LSL)-Kras^{G12D} (abbreviated “K”, see Johnson et al., 2001), Lkb1^{L/L} (see Bardessy et al., 2002), and p53^{L/L} (see Jonkers et al., 21001) were intercrossed. Derivative cells from these crosses were used to study melanocyte growth and melanomagenesis in vitro and in vivo.

To investigate the effect of Lkb1 on melanocyte growth and proliferation, murine melanocytes from neonatal mice of defined genotypes were isolated. Melanocytic origin of the cells was confirmed by immunofluorescence staining for the expression of tyrosinase and tyrosinase-related protein 1. Cells were treated with 4-OHT in vitro to allow CRE activation and induce allelic recombination, which was confirmed by PCR. While wild-type (WT), TK, TLkb1^{L/L} and 4-OHT-untreated control melanocytes grew poorly in vitro, 4-OHT-treated primary melanocytes from LKLkb1^{L/L} mice demonstrate robust in vitro proliferation with no detectable growth arrest over two months. See FIG. 1. Ink4a/Arf-deficient melanocytes are similarly immortal in culture (see Sviderskaya et al., 2002), and these findings suggest Lkb1 function is required for Ras-mediated Ink4a/Arf activation in melanocytes as is the case in murine embryo fibroblasts. See Bardessy et al., 2002.

To examine the role of Lkb1 in melanocytes in vivo, neonatal mice were topically treated with 4-OHT to activate CRE and induce recombination as previously described. See Bosenberg et al., 2006 and Monahan et al., 2010. Within four weeks of 4-OHT treatment, mice from K-Ras expressing cohorts (TK, TKLkb1^{L/L}, TKp53^{L/L} and TKp53^{L/L}Lkb1^{L/L}) developed melanocytic hyperproliferation and exhibited pigmented macules in the skin, while wild-type or 4-OHT-untreated littermates appeared normal. The effects were stronger in TKLkb1^{L/L} and TKp53^{L/L} cohorts than in the TK cohort, and the most pronounced effects were observed in TKp53^{L/L}Lkb1^{L/L} mice. Accompanying the obvious melanocytic hyperproliferation in the tails and paws, coat color was significantly more heterogeneous and darker when K-Ras activation was combined with Lkb1 loss. The skin and coat color from K-Ras wild-type cohorts (TLkb1^{L/L}, Tp53^{L/L}, and Tp53^{L/L}Lkb1^{L/L}) appeared normal. In aggregate, these in vitro and in vivo data appear to show that homozygous Lkb1 inactivation is not sufficient to induce melanocytic hyperproliferation in isolation, but potently cooperates with somatic K-Ras activation (+/−p53 loss) in this regard.

### Example 3

### Lkb1 Inactivation Promotes Melanoma Formation and Metastasis

To characterize the role of Lkb1 in melanomagenesis, the effects of Lkb1 loss in K-Ras-induced melanoma was studied using a previously described system to look at somatic, melanocyte-specific tumor suppressor inactivation. See Monahan et al., 2010. Tumors were not observed in TK mice, nor in animals of any genotype without K-Ras activation (TLkb1^{L/L}, Tp53^{L/L}, or Tp53^{L/L}Lkb1^{L/L}) when followed to 70 weeks. See FIG. 2. Combined somatic Lkb1 loss and K-Ras activation, however, led to melanoma formation with 100% penetrance and latencies ranging from 24 to 56 weeks (median of 38.5). As previously reported (see Monahan et al., 2010), concomitant somatic p53 deletion combined with K-Ras activation also potently facilitated tumorigenesis, with a penetrance and median latency similar to that seen in the TKLkb1^{L/L} mice. Despite suggestions that Lkb1 loss compromises p53 function (see Jones et al., 2005; Karuman et al., 2001; and Zeng and Berger, 2006), strong cooperation between deletion of Lkb1 and p53 in the context of K-Ras activation (TKp53^{L/L}Lkb1^{L/L}) was noted, with a sharp reduction of median tumor latency to 11 weeks. Therefore, in accord with prior observations in murine lung tumors (see Ji et al., 2007), Lkb1 and p53 independently restrain Ras-mediated tumorigenesis in vivo.

TABLE 4
Tumor Formation and Metastasis by Genotype and Site in
Genetically-Engineered Murine (GEM) Models of Melanoma.

| | Primary | | | | | | |
|---|---|---|---|---|---|---|---|
| | Tumors/Treated | # Metastases by Site | | | | | |
| Genotype | Mice | L.N. | Lung | Liver | Spleen | Kidney | Brain |
| TK | 0/12 | 0 | 0 | 0 | 0 | 0 | 0 |
| TKpl6^{L/L} | 8/11 | 0 | 0 | 0 | 0 | 0 | 0 |
| TKp53^{L/L} | 8/11 | 0 | 0 | 0 | 0 | 0 | 0 |
| TKp53^{L/L} p16^{L/L} | 15/15 | 0 | 0 | 0 | 0 | 0 | 0 |
| TKLkb1^{L/L} | 12/12 | 12 | 2 | 2 | 3 | 0 | 0 |
| TKp53^{L/L} Lkb1^{L/L} | 15/15 | 15 | 5 | 3 | 4 | 0 | 0 |

L.N. = lymph node

Although metastasisis is seen with multi-copy N-Ras and c-Met transgenic alleles combined with germline Ink4a/Arfloss (see Ackermann et al., 2005; and Scott et al., 2011), metastasis is not a feature of melanoma models driven by a multi-copy H-Ras transgenic allele (see Chin et al., 1997; and Scott et al., 2011) or the expression of endogenous levels of mutant K-Ras (see Monahan et al., 2010) when combined with somatic p16^{INK4a} or germline Ink4a/Arf loss. To emphasize this point, 300 tumor-bearing melanoma mice resulting from overexpression of mutant H-Ras with Ink4a/Arf loss (“TRIA” mice, see Chin et al., 1997) were followed, and metastasis in mice of this background was never noted. Likewise, hematogenous or lymph node metastases were not observed in K-Ras-driven melanoma models with intact Lkb1 function, including TKp16^{L/L}, TKp53^{L/L}, and TKp53^{L/L}p16^{L/L} mice. See Table 4, above. See also, Monahan et al., 2010. Against this prior experience, it was surprising to note high-volume metastasis in 100% of tumor-bearing mice with somatic K-Ras activation and Lkb1 loss (TKLbk1^{L/L} and TKp53^{L/L}Lkb1^{L/L}). In these mice, metastases were found in lymph node, lung, liver and spleen, but not in kidney or brain. Since metastatsis in Lkb1-intact tumors induced by activated H- or K-Ras (e.g., with combined Ink4a/Arf or p53 loss) was not observed, the data is suggestive that the strong enhancement of metastasis in this model resulted from Lkb1 inactivation.

Interestingly, while the primary melanomas in both TKLkb1^{L/L} and TKp53^{L/L}Lkb1^{L/L} mice were unpigmented or hypopigmented, metastases found in lymph node, lung, liver and spleen contained both unpigmented and deeply pigmented lesions. Therefore, without being bound to any one theory, loss of Lkb1 appears to strongly promote melanoma metastasis in the context of increased tumor heterogeneity and differentiation potential, consistent with an effect of Lkb1 on a tumor-initiating compartment with increased multipotency.

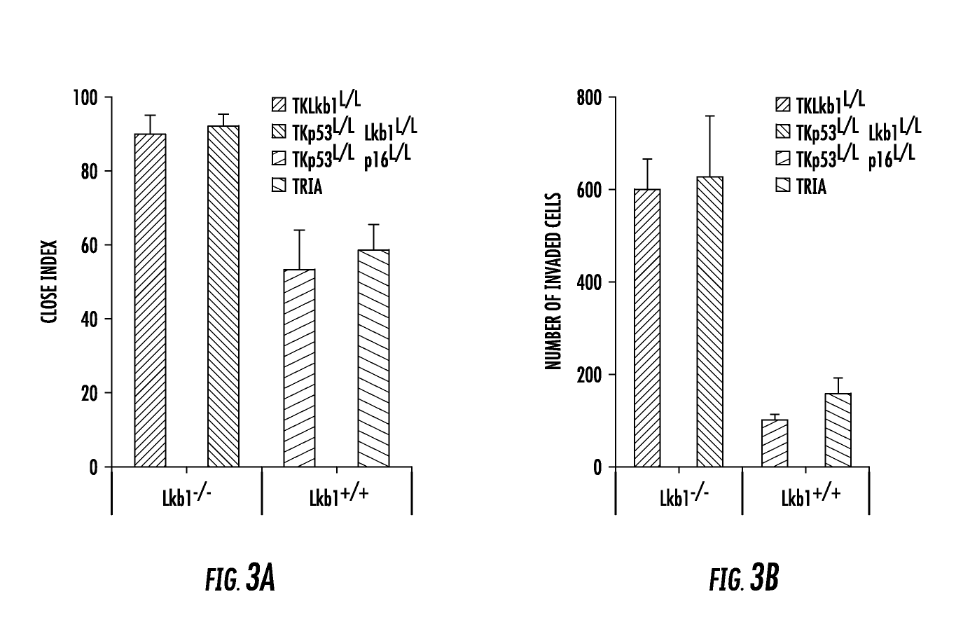

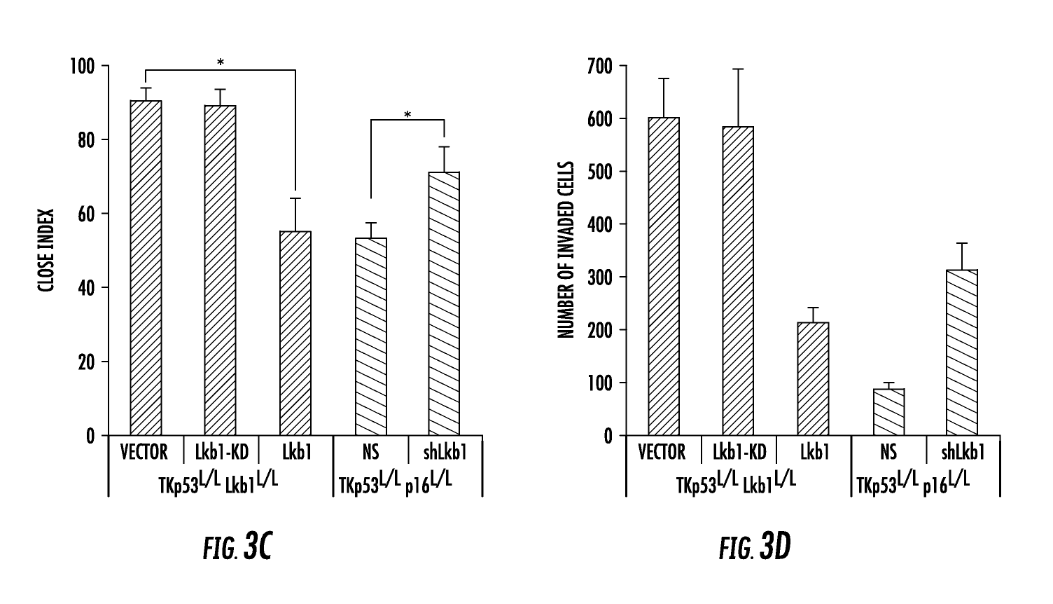

To further understand the mechanism whereby Lkb1 regulates metastasis, the effects of Lkb1 on cell migration and invasion were studied in vitro. Tumor cell lines were generated from mice of defined genotypes with and without Lkb1. Lkb1 loss appeared to have a strong effect as determined by in vitro wound healing or scratch assay. Compared to melanoma cells with wild-type Lkb1, including Tkp53^{L/L}p16^{L/L} and TRIA cells, Lkb1-deficient melanoma cells migrated more rapidly to fill an in vitro wound. See FIG. 3A. Likewise, loss of Lkb1 increased tumor invasiveness as quantified using the matrigel invasion assay. See FIG. 3B. To confirm that these effects reflected Lkb1 function, Lkb1 expression was restored in Lkb1-null melanoma cells by transducing wild-type Lkb1 or kinase-dead Lkb1 (Lkb1-KD), and Lkb1 expression in Lkb1 intact melanoma cell lines was knocked down by transducing an shRNA targeting Lkb1. In scratch assays and matrigel invasion, Lkb1 restoration in Lkb1-null tumor cells inhibited cell migration and invasion, which was dependent on the kinase activity of Lkb1. Likewise a partial knockdown of Lkb1 in TKp53^{L/L}p16^{L/L} cell lines significantly promoted cell migration and invasion. See FIGS. 3C and 3D. These data demonstrate that loss of Lkb1 promotes melanoma cell migration and invasion in vitro.

### Example 4

### Lkb1 Loss Results in SRC-Family Kinase (SFK) Activation

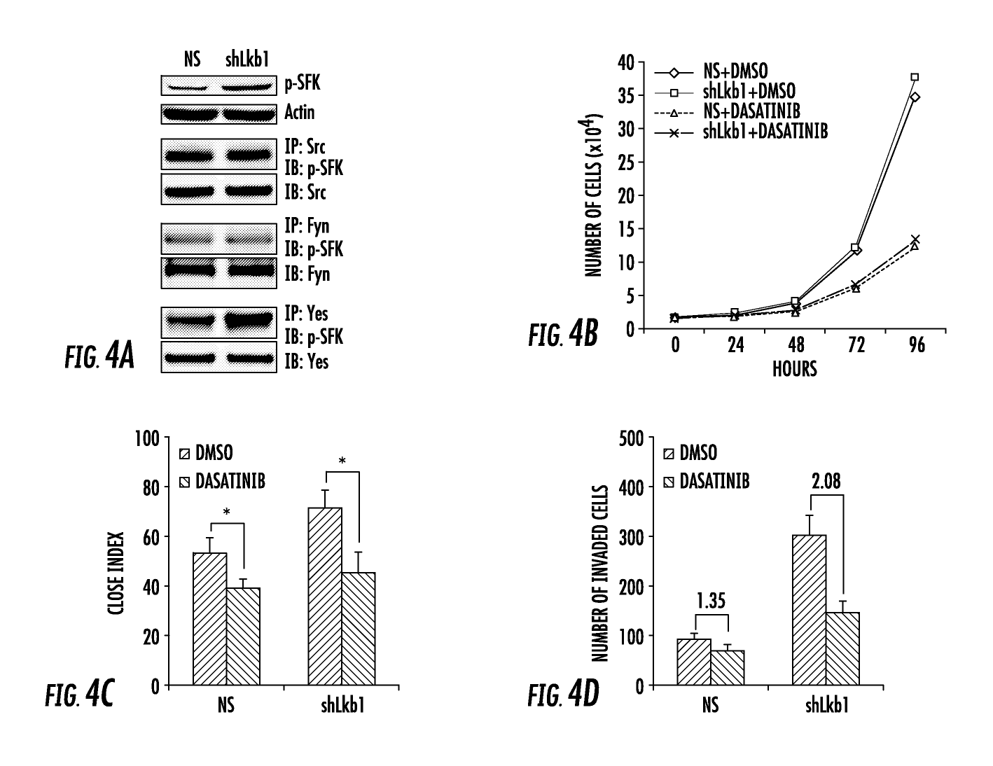

Unbiased proteomic analysis has revealed that Lkb1 loss activates SFKs in lung tumors. See Carretero et al., 2010. Therefore, the effect of Lkb1 function on SFKs phosphorylation (which correlates with SFKs activation) in melanoma cells was examined. Lkb1 knockdown led to increased phosphorylation of SFKs in murine TKp53^{L/L}p16^{L/L} melanoma cells using a pan-SFK phospho-specific antibody. See FIG. 4A. The phosphorylation states of individual SFKs members that are abundantly expressed in melanoma, including Src, Fyn, and Yes were also examined by immunoprecipitation of each protein with an SFK-specific antibody followed by immunoblotting with an antibody that recognized a shared phospho-tyrosine site (Y416). While Src and Fyn phosphorylation were not significantly changed by Lkb1 knockdown, Yes phosphorylation was significantly increased by Lkb1 knockdown in melanoma cells. These data suggest that Yes activity, at least in part, reflects Lkb1 function in melanoma.

To test whether increased SFK activity is involved in the effect of LKB1 loss on melanoma cells, TKp53^{L/L}p16^{L/L} melanoma cells with or without LKB1 knockdown were treated with the pan-SFK inhibitor dasatinib. Dasatinib treatment significantly inhibited melanoma cell proliferation. See FIG. 4B. However, the effect was independent of Lkb1 knockdown. In contrast, while treatment with dasatinib resulted in a modest decrease (14%) in cell migration in Lkb-intact melanoma cells, the effect was enhanced (27%) in melanoma cells with Lkb1 knockdown. See FIG. 4C. A similar Lkb1-dependent effect of dasatinib on cell invasion was noted in matrigel invasion. See FIG. 4D. These observations suggest that the activation of SFKs due to Lkb1 loss contributes to melanoma cell migration and invasion, but not proliferation.

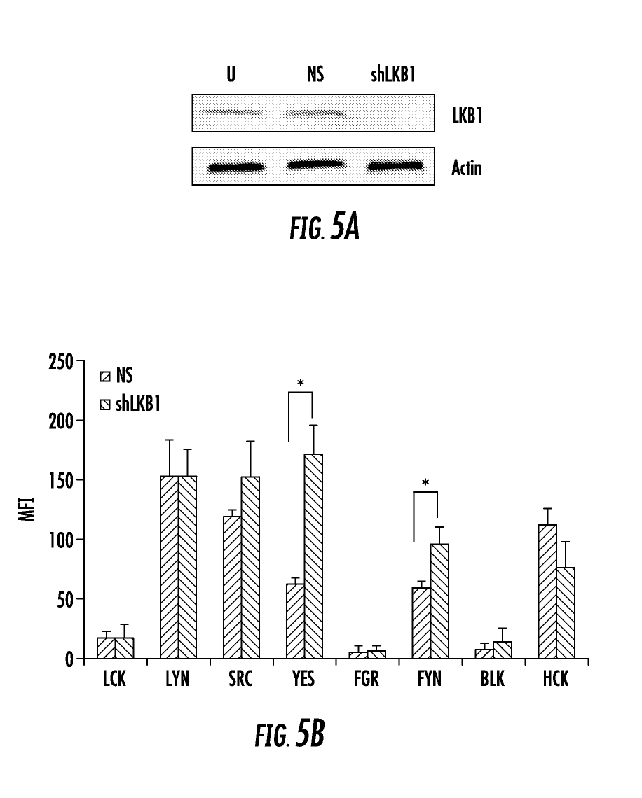

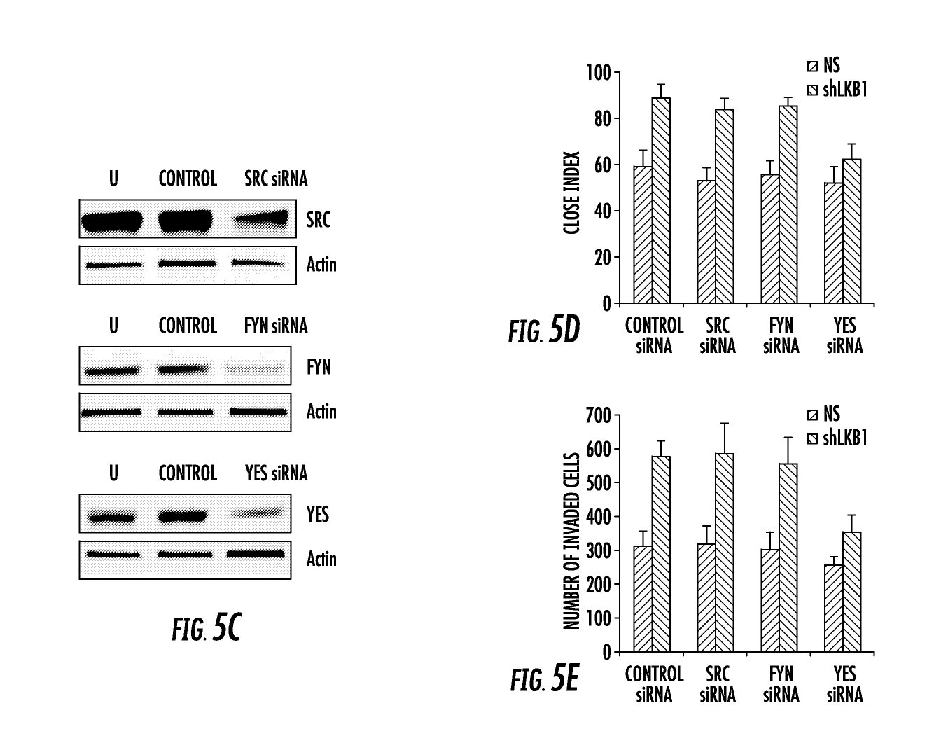

To confirm the effects of LKB1 loss and SFK activity across species, human melanoma cell lines were studied. It was previously noted that expression of LKB1 is highly heterogeneous among a panel of 11 human cell lines. See Rozenberg et al., 2010. Consistent with other experience in trying to decrease kinase activity through shRNA expression, little phenotype was observed in cell lines that highly expressed LKB1 where incomplete knockdown was accomplished. A B-RAF mutant, RB-null melanoma cell line (A2058) was noted to have relatively low expression of LKB1 in the context of a heterozygous coding mutation. Therefore, near complete knock down of LKB1 could be achieved in these cells. See FIG. 5A. The phosphorylation status of all SFKs in the setting of LKB1 knockdown was analyzed using an 8-plex Luminex™ bead assay. In accordance with the murine results (see FIG. 4A), LKB1 knockdown in human A2058 cells resulted in a substantial increase in YES phosphorylation, as well as a more modest but significant effect on FYN phosphorylation. See FIG. 5B. The activity of all the other SFK members was not significantly changed by LKB1 knockdown. See FIG. 5B.

To assess the role of individual SKFs in mediating the effects of LKB1 loss, the expression of individual SFK members was efficiently knocked down by transfecting A2058 cells with siRNAs specifically targeting SRC, FYN, or YES. See FIG. 5C. LKB1 knockdown in A2058 cells had a similar effect on would healing and matrigel invasion to that seen in murine melanoma cells. See FIGS. 5D and 5E. This effect of LKB1 inactivation was reverted by knockdown of YES, but not FYN or SRC. See FIGS. 5D and 5E. Therefore, whereas in lung cancer, a greater effect was seen on SRC (see Carretero et al., 2010), the effects of increased SFK activity on cell migration and invasion associated with LKB1 loss in melanoma cells appears to be predominantly mediated by the YES SFK.

### Example 5

### Lkb1 Loss Expands a Pro-Metastatic Cd24+ Cell Population

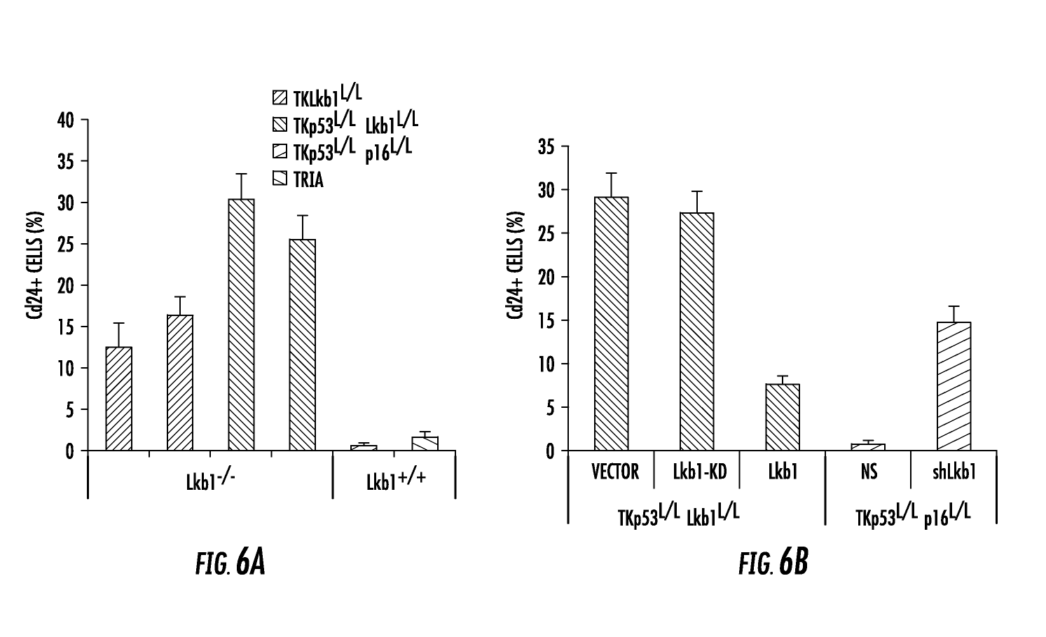

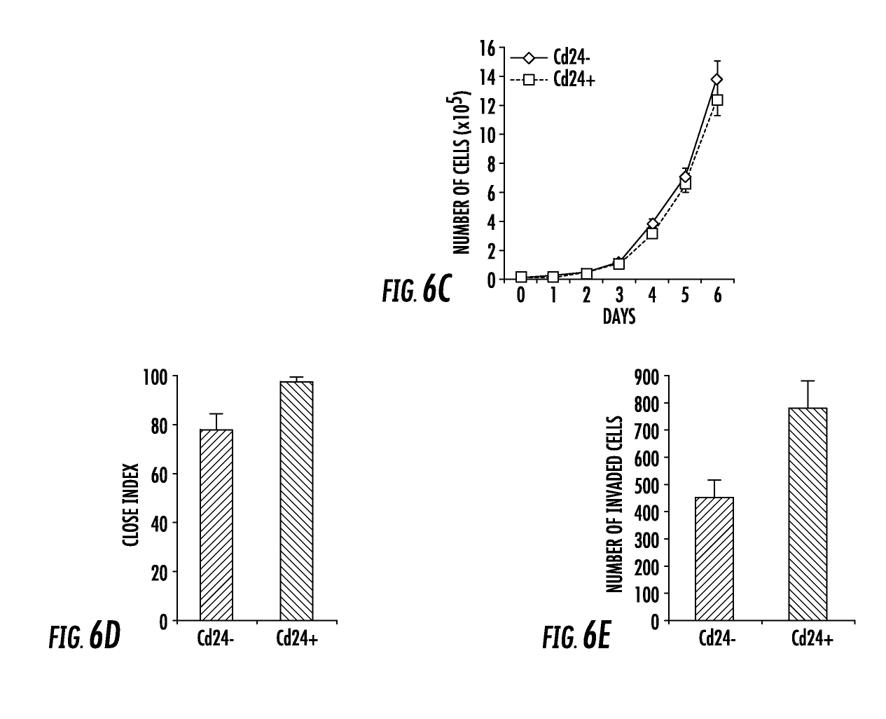

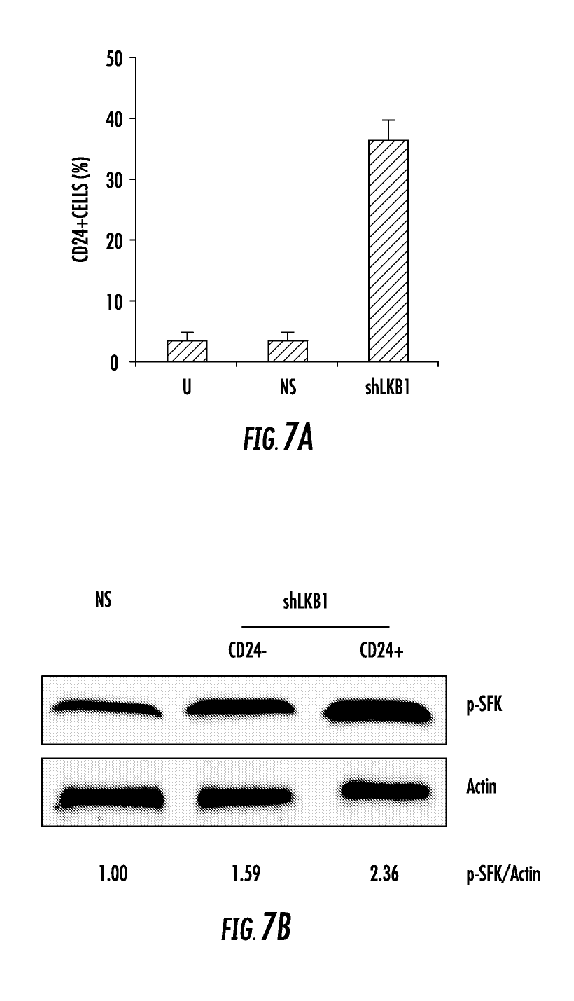

Using an unbiased RNA microarray analysis, it has previously been shown shown that LKB1 regulates expression of CD24 message and protein in human and murine lung tumors. See Ji et al., 2007. Additionally, heterogeneous expression of CD24 in human melanoma cell lines and primary tumors has been demonstrated. See Shields et al., 2007; and Stuelten et al., 2010. CD24 expression is not uniform within a given melanoma cell line, but rather is generally expressed on a tumor sub-fraction, with expression ranging from <1% to 13% of cells. Given that CD24 is a known modulator of advanced disease and metastasis (see Baumann et al., 2005; Kristiansen et al., 2003a; Kristiansen et al., 2003b; Lee et al., 2011; Senner et al., 1999; and Weichert et al., 2005) and a marker of stem-progenitor cells in several tumor types (see Al-Hajj et al., 2003; Gao et al., 2010; Hurt et al., 2008; Lee et al., 2011; and Li et al., 2007), the effect of LKB1 on CD24 expression was examined in murine melanoma. Cell lines derived from murine melanomas with intact Lkb1 function exhibited a low fraction (<3%) of Cd24⁺ cells. Inactivation of Lkb1 was associated with a marked expansion of the Cd24⁺ population, ranging from 10% to more than 30% of cells. See FIGS. 6A and 6B. Correspondingly, restored expression of Lkb1 in Lkb1-null melanoma cells suppressed Cd24 expression withing 6 days of transduction, which was dependent on the kinase activity of Lkb1. See FIG. 6B. These data demonstrate a highly dynamic, 3-10-fold effect of Lkb1-kinase activity on expression of cell surface Cd24, a known facilitator of metastasis.

Given that Cd24 expression (both increased and decreased) has been associated with functional heterogeneity and tumor-initiating cells in other cancer types (see Al-Hagg et al., 2003; Gao et al., 2010; Hurt et al., 2008; Lee et al., 2011; and Li et al. 2007), the in vitro properties of Cd24⁺ vs. Cd24⁻ cells in melanoma cell lines was examined. Cd24⁺ and Cd24⁻ cells were isolated from TKp53^{L/L}Lkb1^{L/L} cells by fluorescence activated cell sorting (FACS), and the separated populations were assessed for proliferation, migration and invasion. No difference was observed in the proliferation of Cd24⁺ versus Cd24⁻ cells. See FIG. 6C. In contrast, Cd24⁺ cells showed increased cell migration and invasion compared to Cd24⁻ cells. See FIGS. 6D and 6E.

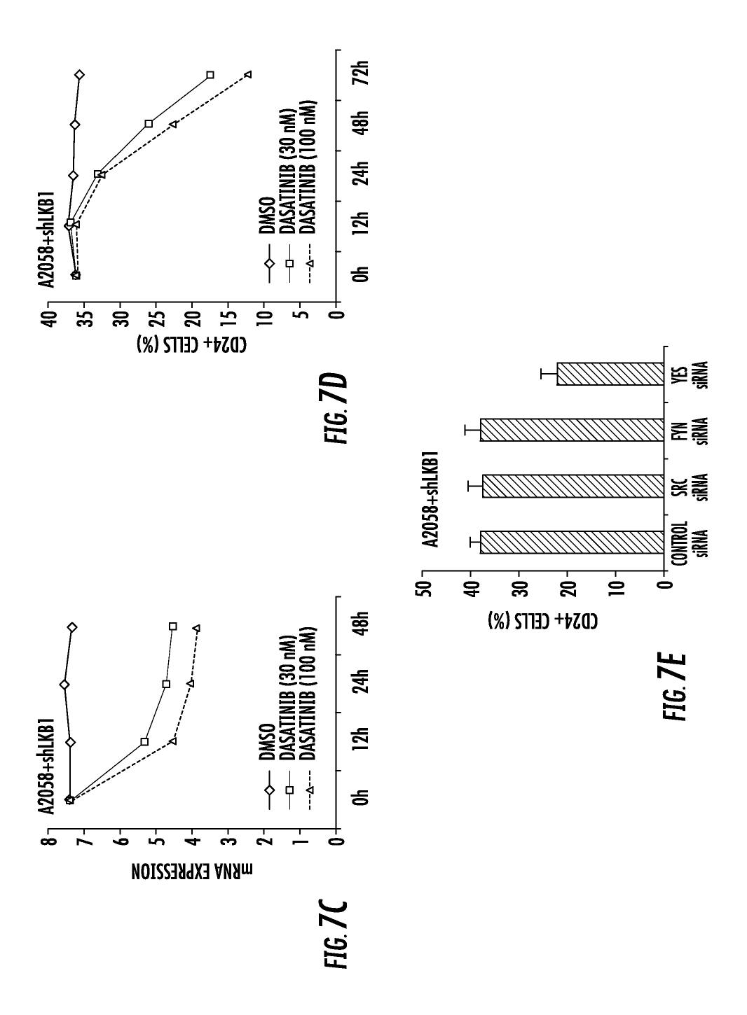

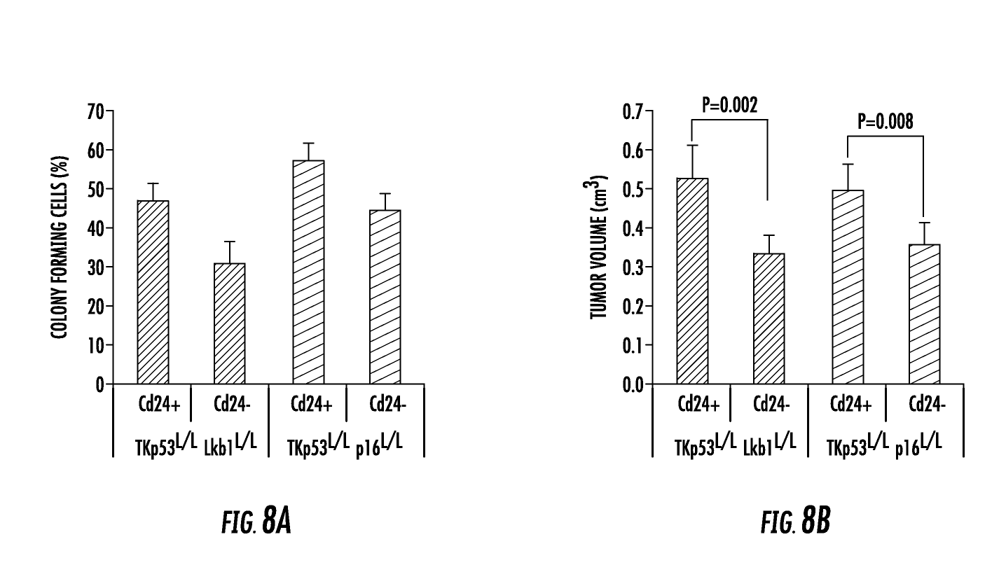

The effects of LKB1 on CD24 expression in human A2058 melanoma cells was also examined. Comparable to other human melanoma cell lines (see Shields et al., 2007; and Stuelten et al., 2010), A2058 cells demonstrate a small fraction (<3%) of CD24⁺ cells. As in the murine system, CD24 expression was markedly and rapidly increased to more than 30% of cells after LKB1 knockdown. See FIG. 7A. Expression of CD44, another commonly used “tumor stem cell” marker, was not modulated by LKB1 knockdown within this time frame. These murine and human cell line data demonstrate that LKB1 kinase activity controls the size of a CK24⁺ sub-fraction in melanoma cell lines that exhibits enhanced metastatic behavior in vitro.

The association of increased SFK activity and CD24 expression with increased cell migration/invasion in LKB1-deficient cells suggested a possible link between SFK signaling and CD24 expression. SFK activity was examined in isolated CD24⁺ and CD24⁻ A2058 cells. See FIG. 7B. Although SFKs activity was moderately increased (1.6-fold) in CD24⁻ cells by LKB1 knockdown, the increase was significantly greater in CD24⁺ cells (2.4-fold). The increase in CD24 mRNA and protein expression due to LKB1 loss was suppressed by transiently treating cells with the pan-SRC inhibitor dasatinib in a dose-dependent fashion in both human and murine melanoma cells (see FIGS. 7C and 7D), with CD24 mRNA sharply decreasing with as little as 12 hours of dasatinib treatment. In accord with the in vitro motility and invasion results (see FIGS. 5D and 5E), the effect of LKB1 loss on CD24 expression was rescued by siRNA to YES, but not SRC or FYN. See FIG. 7E. These data show that the ability of LKB1 loss to induce expansion of the pro-metastatic CD24⁺ compartment requires the activity of SKFs, specifically YES kinase.

To determine if the effects of LKB1 loss were primarily via modulation of the size of the CD24⁺ compartment or if LKB1 loss conferred increased metastatic behavior in all melanoma cells regardless of CD24 status, the in vitro progenitor abilities of CD24⁺ cells were measured in both Lkb1-deficient (TKp53^{L/L}Lkb1^{L/L}) and Lkb1-competent (TKp53^{L/L}p16^{L/L}) lines by performing colony forming assays with sorted Cd24⁺ and Cd24⁻ cells. See FIG. 8A. The abundance of colony forming cells (CFCs) was more abundant and to the same degree in the Cd24⁺ fractions from both Lkb1-null and Lkb1-competent cells.

The in vivo tumor growth of Cd24⁺ and Cd24⁻ cells was investigated by xenograft transplantation. Cd24⁺ cells and Cd24⁻ cells were isolated by FACS and injected into nude mice. Although all mice developed tumors within three weeks of injection, Cd24⁺ cells grew more rapidly and to larger tumor volumes. See FIG. 8B. The Cd24⁺ fractions demonstrated a comparable enhancement of tumor growth whether they were derived from Lkb1-defective or competent melanomas. These in vitro and in vivo data indicated that Lkb1 inactivation promotes tumor progression predominantly by leading to a marked expansion of a Cd24⁺ fraction that demonstrates increased invasive and progenitor properties.

### Example 6

### Discussion of Examples 1-5

As demonstrated above, mice with melanocyte-specific Lkb1 loss and K-Ras activation develop penetrant and highly metastatic melanomas. Lkb1-deficient melanoma cells demonstrate increased invasive behavior in vitro compared to isogenic Lkb1-competent melanoma cells. Further, LKB1 deficiency results in activation of SRC-family kinases (SFKs), particularly YES, and expansion of a CD24⁺ cell population that shows increased invasive behavior both in vitro and in vivo. Genetic or pharmacologic inhibition of YES activity suppresses CD24 expression and decreases metastatic behavior. Collectively, these results demonstrate that LKB1 functions as a strong suppressor of melanoma metastasis by regulating YES activity which determines the size of a pro-metastatic CD24⁺ tumor sub-population.

Of interest with regard to the phenotypic expression of PJS, the combined melanocyte-specific Lkb1 loss and K-Ras activation results in increased melanocyte proliferation and in vivo hyperpigmentation. The excess melanocytic proliferation in TKLkb1^{L/L} mice (and even TKLkb1^{L/+}), but not in TLkb1^{L/L} or Tp53^{L/L}Lkb1^{L/L} mice, suggests that mucocutaneous melanocytic hyperproliferation seen in PJS patients can reflect sporadic secondary events that activate regulators of proliferation such as RAS rather than loss of heterozygosity (LOH) of the second copy of LKB1. Thus, the Lkb1-deficient mouse model described herein appears to serve as a model to study this poorly understood feature of PJS syndrome.

In addition to altered pigmentation, TKLkb1^{L/L} and TKp53^{L/L}Lkb^{L/L} mice exhibit highly metastatic melanoma. Although metastasis has been reported in a small number of autothchonous murine tumor models (e.g., N-Ras or c-Met Ink4a/Arf−/− transgenic melanomas (see Ackermann et al., 2005; and Scott et al., 2011) and Polyoma middle T breast cancer (see Guy et al., 1992)), in general these models feature considerably lower volumes of metastatic disease with variable penetrance and rely on supra-physiologic expression of oncogenes. In contrast, the presently disclosed model couples melanocyte-specific, somatic single-copy K-Ras activation under the control of its endogenous promoter with homozygous Lkb1 deletion to produce 100% penetrance of metastasis with a high burden of metastatic disease. For example, several tumor-bearing TKLkb1^{L/L} and TKLkb1^{L/L}p53^{L/L} mice exhibited >50% involvement of the liver, lung and/or spleen with multi-focal metastasis of variable histology and pigmentation. Thus, the high burden and penetrance of metastases in the presently disclosed model can address a unmet need in cancer research of experimentally tractable, highly metastatic autochthonous tumor models.

Although LKB1 has been reported to function through activation of p53, p16^{INK4a} and/or Arf (see Bardessy et al., 2002; and Karuman et al., 2001), the presently disclosed data indicate that LKB1 also effects p53- and Ink4a/arf-independent tumor suppressor roles. In murine models of both lung cancer (see Carretero et al., 2010; and Ji et al., 2007) and melanoma, Lkb1-deficient tumors demonstrate increased histomorphometric heterogeneity and more frequent metastasis compared to tumors lacking p53 or Ink4a/Arf, and p53 deficiency strongly cooperates with Lkb1 loss to shorten tumor latency. Melanoma metastasis, albeit with lower burdens, has been reported in 4-OHT-treated Tyr-CRE-ER^{T2}B-Raf^{LSL/+}Pten^{L/L} mice. See Dankort et al., 2009. This is consistent with the notion that either B-Raf mutation (see Esteve-Puig et al., 2009; and Zheng et al., 2009) or Pten loss (see Huang et al., 2008) induces a partial compromise of Lkb1 function. However, with regard to metastasis, the phenotype of TKLkb1^{L/L} mice appears stronger than any of these other models, which, without being bound to any one theory, suggest that loss of any of these other tumor suppressors (Pten, p53, p16INK4a, or Arf) or B-Raf activation is not entirely redundant with Lkb1 deficiency.

As described herein, LKB1 loss results in YES activation, and genetic or pharmacologic inhibition of YES activity suppresses the effects of LKB1 loss on enhancing cell metastatic properties. Although the mechanism whereby loss of LKB1 kinase activity induces YES activation is not known, the data identify YES as a new therapeutic target in melanoma lacking LKB1 function. Along these lines, it has recently been reported that tumor regression is seen in 17% (6 of 36) of patients with advanced melanoma in response to the treatment with dasatinib. See Kluger et al., 2011. Dasatinib response in this series did not correlate with activating mutation of c-Kit (a known driver in a small fraction of human melanoma), suggesting that determination of LKB1 mutation status can help to predict dasatinib response in human patients.

Increased YES activity in turn leads to an expansion of a tumor sub-population that is characterized by increased cell motility and invasion, as well as CD24⁺ expression. Surprisingly, although LKB1 function is inhibited in most or all of the cells, the activation of YES and expression of CD24 in response to LKB1 inactivation is limited to a minority (−10-30%) of cells, which exhibit enhanced metastatic properties. This finding cannot represent variable knockdown by RNA interference (RNAi), since an identical finding is seen using genetic Lkb1 deficiency (i.e. in TKLkb1^{L/L} lines). A CD24⁺ population of cells is present, albeit at considerably lower frequency, in LKB1-competent melanoma cells, and loss of LKB1 kinase activity appears to induce an expansion of this pro-metastatic fraction.

In colony forming and xenograft assays, the pro-metastatic properties of CD24⁺ cells were increased relative to isogenic CD24⁻ cells regardless of whether the CD24⁺ cells were derived from LKB1-deficient or -competent cell lines. This observation is in accordance with the evidence that CD24 expression is associated with advanced disease and increased metastasis in glioma and many epithelial cancers. See Baumann et al., 2005; Kristiansen et al., 2003a; Kristiansen et al., 2003b; Lee et al., 2011; Senner et al., 1999; and Weichert et al., 2005. Therefore, the presently disclosed data are most consistent with the model that the principal effect of LKB1 inactivation with regard to metastasis is to markedly increase the frequency of this pro-metastatic sub-population.

While CD24 expression appears to play a direct role in facilitating tumor metastasis, it has also been observed to mark heterogeneous sub-populations (e.g. ‘tumor stem cells’) of a variety of cancers. See Al-Hail et al., 2003; Gao et al., 2010; Hurt et al., 2008; Lee et al., 2011; and Li et al., 2007. Therefore, the presently disclosed data are believed to be consistent with the model that CD24 expression directly facilitates melanoma metastasis, but also that CD24 expression merely serves as a marker of a tumor sub-population with increased metastatic properties. With regard to the latter possibility, LKB1 loss leads to an increase in a tumor sub-fraction with increased colony forming activity and expanded tumor differentiation potential in vivo (as reflected by the variable degree of tumor pigmentation), which are properties of ‘tumor stem cells’. While the concept of a tumor stem cell in melanoma is controversial (see Quintana et al., 2010; Quintana et al., 2008; and Roesch et al., 2010), the presently disclosed results are compatible with possibility that the increased tumor heterogeneity noted the setting of LKB1 inactivation reflects an augmented tumor stem cell fraction.

In summary, the presently disclosed subject matter shows a prominent role for LKB1 in melanocyte biology and the suppression of melanoma metastasis. A principal effect of LKB1 loss on metastasis requires expansion of a CD24⁺ pro-metastatic tumor sub-fraction that exhibits some properties of a tumor stem cell. Expansion of this compartment requires the activity of YES kinase. Without being bound to any one theory, these data suggest that a determination of LKB1 mutational status in patients with advanced melanoma can contribute to prognosis prediction, and identifies novel therapeutic targets (YES and CD24) in the substantial fraction of melanoma lacking LKB1 function.

### REFERENCES

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
Ackermann, J., Frutschi, M., Kaloulis, K., McKee, T., Trumpp, A., and Beermann, F. (2005). Metastasizing melanoma formation caused by expression of activated NRasQ61K on an INK4a-deficient background. Cancer Res, 65, 4005-4011.
Albert et al. (1992). J Virol, 66, 5627-5630.
Alexay et al. (1996). Fluorescence scanner employing a macro scanning objective. SPIE Proceedings, 2705, 63-270.
Al-Hajj, M., Wicha, M. S., Benito-Hernandez, A., Morrison, S. J., and Clarke, M. F. (2003). Prospective identification of tumorigenic breast cancer cells. Proc Natl Acad Sci USA, 100, 3983-3988.
Alessi, D. R., Sakamoto, K, and Bayascas, J. R. (2006). LKB1-dependent signaling pathways. Annu Rev Biochem, 75, 137-163.
Ausubel et al. (2002). Short Protocols in Molecular Biology, Fifth ed. John Wiley & Sons, Inc., New York, N.Y.
Ausubel et al. (2003). Current Protocols in Molecular Biology, John Wiley and Sons, Inc., New York, N.Y.
Bardeesy, N., Sinha, M., Hezel, A. F., Signoretti, S., Hathaway, N. A., Sharpless, N. E., Loda, M., Carrasco, D. R., and DePinho, R. A. (2002). Loss of the Lkb1 tumour suppressor provokes intestinal polyposis but resistance to transformation. Nature, 419, 162-167.
Baumann, P., Cremers, N., Kroese, F., Orend, G., Chiquet-Ehrismann, R., Uede, T., Yagita, H., and Sleeman, J. P. (2005). CD24 expression causes the acquisition of multiple cellular properties associated with tumor growth and metastasis. Cancer Res, 65, 10783-10793.
Bej et al. (1991). Appl Environ Microbiol, 57, 3529-3534.
Bennett, D. C., Cooper, P. J., Dexter, T. J., Devlin, L. M., Heasman, J., and Nester, B. (1989). Cloned mouse melanocyte lines carrying the germline mutations albino and brown: complementation in culture. Development (Cambridge, England), 105, 379-385.
Boom et al. (1990). J. Clin Microbiol, 28, 495-503.
Bosenberg, M., Muthusamy, V., Curley, D. P., Wang, Z., Hobbs, C., Nelson, B., Nogueira, C., Horner, J. W., 2nd, Depinho, R., and Chin, L. (2006). Characterization of melanocyte-specific inducible Cre recombinase transgenic mice. Genesis, 44, 262-267.
Buffone et al. (1991). Clin Chem, 37, 1945-1949.
Busch et al. (1992). Transfusion, 32, 420-425.
Carretero, J., Shimamura, T., Rikova, K., Jackson, A. L., Wilkerson, M. D., Borgman, C. L., Buttarazzi, M. S., Sanofsky, B. A., McNamara, K. L., Brandstetter, K. A., et al. (2010). Integrative genomic and proteomic analyses identify targets for Lkb1-deficient metastatic lung tumors. Cancer Cell, 17, 547-559.
Cha and Thilly (1993). PCR Methods Appl, 3, S18-S29.
Cheng, H., Liu, P., Wang, Z. C., Zou, L., Santiago, S., Garbitt, V., Gjoerup, 0. V., Iglehart, J. D., Miron, A., Richardson, A. L., et al. (2009). SIK1 couples LKB1 to p53-dependent anoikis and suppresses metastasis. Sci Signal, 2, ra35.
Chin, L., Pomerantz, J., Polsky, D., Jacobson, M., Cohen, C., Cordon-Cardo, C., Homer, J. W., 2nd, and DePinho, R. A. (1997). Cooperative effects of INK4a and ras in melanoma susceptibility in vivo. Genes & development, 11, 2822-2834.
Chiodi et al. (1992). J. Clin Microbiol. 30, 255-258.
Dankort, D., Curley, D. P., Cartlidge, R. A., Nelson, B., Karnezis, A. N., Damsky, W. E., Jr., You, M. J., DePinho, R. A., McMahon, M., and Bosenberg, M. (2009). Braf(V600E) cooperates with Pten loss to induce metastatic melanoma. Nat Genet, 41, 544-552.
DeRisi et al. (1996) Nat Genet, 14, 457-460.
Esteve-Puig, R., Canals, F., Colome, N., Merlino, G., and Redo, J. A. (2009). Uncoupling of the LKB1-AMPKalpha energy sensor pathway by growth factors and oncogenic BRAF. PLoS One, 4, e4771.
Forbes, S. A., Bindal, N., Bamford, S., Cole, C., Kok, C. Y., Beare, D., Jia, M., Shepherd, R., Leung, K., Menzies, A., et al. (2011). COSMIC: mining complete cancer genomes in the Catalogue of Somatic Mutations in Cancer. Nucleic Acids Res, 39, D945-950.
Gao, M. Q., Choi, Y. P., Kang, S., Youn, J. H., and Cho, N. H. (2010). CD24+ cells from hierarchically organized ovarian cancer are enriched in cancer stem cells. Oncogene, 29, 2672-2680.
Giardiello, F. M., Brensinger, J. D., Tersmette, A. C., Goodman, S. N., Petersen, G. M., Booker, S. V., Cruz-Correa, M., and Offerhaus, J. A. (2000). Very high risk of cancer in familial Peutz-Jeghers syndrome. Gastroenterology, 119, 1447-1453.
Giardiello, F. M., Welsh, S. B., Hamilton, S. R., Offerhaus, G. J., Gittelsohn, A. M., Booker, S. V., Krush, A. J., Yardley, J. H., and Luk, G. D. (1987). Increased risk of cancer in the Peutz-Jeghers syndrome. N Engl J Med, 316, 1511-1514.
Guedon et al. (2000). Anal Chem, 72, 6003-6009.
Guervos, M. A., Marcos, C. A., Hermsen, M., Nuno, A. S., Suarez, C., and Llorente, J. L. (2007). Deletions of N33, STK11 and TP53 are involved in the development of lymph node metastasis in larynx and pharynx carcinomas. Cell Oncol, 29, 327-334.
Guldberg, P., thor Straten, P., Ahrenkiel, V., Seremet, T., Kirkin, A. F., and Zeuthen, J. (1999). Somatic mutation of the Peutz-Jeghers syndrome gene, LKB1/STK11, in malignant melanoma. Oncogene, 18, 1777-1780.
Guy, C. T., Cardiff, R. D., and Muller, W. J. (1992). Induction of mammary tumors by expression of polyomavirus middle T oncogene: a transgenic mouse model for metastatic disease. Mol Cell Biol, 12, 954-961.
Hamel et al. (1995). J Clin Microbiol, 33, 287-291.
Heaton et al. (2001) Proc Natl Acad Sci USA, 98, 3701-3704.
Hemminki, A., Markie, D., Tomlinson, I., Avizienyte, E., Roth, S., Loukola, A., Bignell, G., Warren, W., Aminoff, M., Hoglund, P., et al. (1998). A serine/threonine kinase gene defective in Peutz-Jeghers syndrome. Nature, 391, 184-187.
Herrewegh et al. (1995). J Clin Microbiol, 33, 684-689.
Huang, X., Wullschleger, S., Shpiro, N., McGuire, V. A., Sakamoto, K., Woods, Y. L., McBurnie, W., Fleming, S., and Alessi, D. R. (2008). Important role of the LKB1-AMPK pathway in suppressing tumorigenesis in PTEN-deficient mice. Biochem J, 412, 211-221.
Hurt, E. M., Kawasaki, B. T., Klarmann, G. J., Thomas, S. B., and Farrar, W. L. (2008). CD44+ CD24(−) prostate cells are early cancer progenitor/stem cells that provide a model for patients with poor prognosis. Br J Cancer, 98, 756-765.
Izraeli et al. (1991). Nuc Acids Res, 19, 6051.
Jeghers, H., Mc, K. V., and Katz, K. H. (1949). Generalized intestinal polyposis and melanin spots of the oral mucosa, lips and digits; a syndrome of diagnostic significance. N Engl J Med, 241, 1031-1036.
Jenne, D. E., Reimann, H., Nezu, J., Friedel, W., Loff, S., Jeschke, R., Muller, 0., Back, W., and Zimmer, M. (1998). Peutz-Jeghers syndrome is caused by mutations in a novel serine threonine kinase. Nat Genet, 18, 38-43.
Ji, H., Ramsey, M. R., Hayes, D. N., Fan, C., McNamara, K., Kozlowski, P., Torrice, C., Wu, M. C. Shimamura, T., Perera, S. A., at al. (2007). LKB1 modulates lung cancer differentiation and metastasis. Nature, 448, 807-810.
Johnson, L., Mercer, K., Greenbaum, D., Bronson, R. T., Crowley, D., Tuveson, D. A., and Jacks, T. (2001). Somatic activation of the K-ras oncogene causes early onset lung cancer in mice. Nature, 410, 1111-1116.
Jones, R. G., Plas, D. R., Kubek, S., Buzzai, M., Mu, J., Xu, Y., Birnbaum, M. J., and Thompson, C. B. (2005). AMP-activated protein kinase induces a p53-dependent metabolic checkpoint. Mol Cell, 18, 283-293.
Jonkers, J., Meuwissen, R., van der Gulden, H., Peterse, H., van der Valk, M., and Berns, A. (2001). Synergistic tumor suppressor activity of BRCA2 and p53 in a conditional mouse model for breast cancer. Nat Genet, 29, 418-425.
Karuman, P., Gozani, 0., Odze, R. D., Zhou, X. C., Zhu, H., Shaw, R., Brien, T. P., Bozzuto, C. D., Ooi, D., Cantley, L. C., and Yuan, J. (2001). The Peutz-Jegher gene product LKB1 is a mediator of p53-dependent cell death. Molecular cell, 7, 1307-1319.
Kluger, H. M., Dudek, A. Z., McCann, C., Ritacco, J., Southard, N., Jilaveanu, L. B., Molinaro, A., and Sznol, M. (2011). A phase 2 trial of dasatinib in advanced melanoma. Cancer, 117, 2202-2208.
Kohsaka and Carson (1994). J Clin Lab Anal, 8, 425-455.
Kristiansen, G., Schluns, K., Yongwei, Y., Denkert, C., Dietel, M., and Petersen, I. (2003a). CD24 is an independent prognostic marker of survival in nonsmall cell lung cancer patients. Br J Cancer, 88, 231-236.
Kristiansen, G., Winzer, K. J., Mayordomo, E., Bellach, J., Schluns, K., Denkert, C., Dahl, E., Pilarsky, C., Altevogt, P., Guski, H., and Dietel, M. (2003b). CD24 expression is a new prognostic marker in breast cancer. Clin Cancer Res, 9, 4906-4913.
Lanciotti et al. (1992). J Clin Microbiol, 30, 545-551.
Lee, T. K., Castilho, A., Cheung, V. C., Tang, K. H., Ma, S., and Ng, I. 0. (2011). CD24(+) Liver Tumor-Initiating Cells Drive Self-Renewal and Tumor Initiation through STAT3-Mediated NANOG Regulation. Cell Stem Cell, 9, 50-63.
Li, C., Heidt, D. G., Dalerba, P., Burant, C. F., Zhang, L., Adsay, V., Wicha, M., Clarke, M. F., and Simeone, D. M. (2007). Identification of pancreatic cancer stem cells. Cancer Res, 67, 1030-1037.
Lim, W., Olschwang, S., Keller, J. J., Westerman, A. M., Menko, F. H., Boardman, L. A., Scott, R. J., Trimbath, J., Giardiello, F. M., Gruber, S. B., et al. (2004). Relative frequency and morphology of cancers in STK11 mutation carriers. Gastroenterology, 126, 1788-1794.
Linz et al. (1990). J Clin Chem Clin Biochem, 28, 5-13.
Lisle et al. (2001). BioTechniques, 30, 1268-1272.
Lockhart et al. (1996). Nature Biotechnology, 14, 1675-1684.
Matsumoto, S., Iwakawa, R., Takahashi, K., Kohno, T., Nakanishi, Y., Matsuno, Y., Suzuki, K., Nakamoto, M., Shimizu, E., Minna, J. D., and Yokota, J. (2007). Prevalence and specificity of LKB1 genetic alterations in lung cancers. Oncogene, 26, 5911-5918.
McCaustland et al. (1991). J Virol Methods, 35, 331-342.
McCall et al. (1996). Proc Natl Acad Sci USA, 93, 13555-13560.
McPherson et al. (1995). PCR 2: A Practical Approach, IRL Press, New York, New York.
Millar et al. (1995). Anal Biochem, 226, 325-330.
Monahan, K. B., Rozenberg, G. I., Krishnamurthy, J., Johnson, S. M., Liu, W., Bradford, M. K., Horner, J., Depinho, R. A., and Sharpless, N. E. (2010). Somatic p16(INK4a) loss accelerates melanomagenesis. Oncogene, 29, 5809-5817.
Natarajan et al. (1994). PCR Methods Appl, 3, 346-350.
Nelson et al. (2001). Anal Chem, 73, 1-7.
Paladichuk (1999). The Scientist, 13, 20-23.
Piétu et al. (1996). Genome Res, 6, 492-503.
Quintana, E., Shackleton, M., Foster, H. R., Fullen, D. R., Sabel, M. S., Johnson, T. M., and Morrison, S. J. (2010). Phenotypic heterogeneity among tumorigenic melanoma cells from patients that is reversible and not hierarchically organized. Cancer Cell, 18, 510-523.
Quintana, E., Shackleton, M., Sabel, M. S., Fullen, D. R., Johnson, T. M., and Morrison, S. J. (2008). Efficient tumour formation by single human melanoma cells. Nature, 456, 593-598.
Randolph and Waggoner (1995). Nuc Acids Res, 25, 2923-2929.
Robertson and Walsh-Weller (1998). Methods Mol Biol, 98, 121-154.
Roesch, A., Fukunaga-Kalabis, M., Schmidt, E. C., Zabierowski, S. E., Brafford, P. A., Vultur, A., Basu, D., Gimotty, P., Vogt, T., and Herlyn, M. (2010). A temporarily distinct subpopulation of slow-cycling melanoma cells is required for continuous tumor growth. Cell, 141, 583-594.
Roux (1995). PCR Methods Appl, 4, S185-S194.
Rowan, A., Bataille, V., MacKie, R., Healy, E., Bicknell, D., Bodmer, W., and Tomlinson, I. (1999). Somatic mutations in the Peutz-Jeghers (LKB1/STKII) gene in sporadic malignant melanomas. J Invest Dermatol, 112, 509-511.
Rozenberg, G. I., Monahan, K. B., Torrice, C., Bear, J. E., and Sharpless, N. E. (2010). Metastasis in an orthotopic murine model of melanoma is independent of RAS/RAF mutation. Melanoma Res, 20, 361-371.
Rupp et al. (1998). BloTechniques, 6, 56-60.
Sambrook and Russell (2001). Molecular Cloning: A Laboratory Manual, 3^{rd} Ed., Cold Spring Harbor Laboratory Press, Cold Spring Harbor, N.Y.
Sanchez-Cespedes, M. (2007). A role for LKB1 gene in human cancer beyond the Peutz-Jeghers syndrome. Oncogene, 26, 7825-7832.
Sapolsky and Lipshutz (1996). Genomics, 33, 445-456.
Schena et al. (1995). Science, 270, 467-470.
Schena et al. (1996). Proc Natl Acad Sci USA, 93, 10614-10619.
Scott, K. L., Nogueira, C., Heffernan, T. P., van Doom, R., Dhakal, S., Hanna, J. A., Min, C., Jaskelioff, M., Xiao, Y., Wu, C. J., et al. (2011). Proinvasion metastasis drivers in early-stage melanoma are oncogenes. Cancer Cell, 20, 92-103.
Senner, V., Sturm, A., Baur, I., Schrell, U. H., Distel, L., and Paulus, W. (1999). CD24 promotes invasion of glioma cells in vivo. J Neuropathol Exp Neurol, 58, 795-802.
Shalon et al. (1996). Genome Res, 6, 639-649.
Sharpless, N. E., Alson, S., Chan, S., Silver, D. P., Castrillon, D. H., and DePinho, R. A. (2002). p16(INK4a) and p53 deficiency cooperate in tumorigenesis. Cancer Res, 62, 2761-2765.
Shields, J. M., Thomas, N. E., Cregger, M., Berger, A. J., Leslie, M., Torrice, C., Hao, H., Penland, S., Arbiser, J., Scott, G., et al. (2007). Lack of Extracellular Signal-Regulated Kinase Mitogen-Activated Protein Kinase Signaling Shows a New Type of Melanoma. Cancer Res, 67, 1502-1512.
Shoemaker et al. (1996). Nat Genet, 14, 450-456.
Silhavy et al. (1984) Experiments with Gene Fusions, Cold Spring Harbor Laboratory Press, Cold Spring Harbor, N.Y.
Smith et al. (1998). Clin Chem, 44, 2054-2056.
Smith (1998). The Scientist, 12, 21-24.
Spanakis, E., Lamina, P., and Bennett, D. C. (1992). Effects of the developmental colour mutations silver and recessive spotting on proliferation of diploid and immortal mouse melanocytes in culture. Development (Cambridge, England), 114, 675-680.
Strain and Chmielewski (2001). BioTechniques, 30, 1286-1291.
Stuelten, C. H., Mertins, S. D., Busch, J. I., Gowens, M., Scudiero, D. A., Burkett, M. W., Hite, K. M., Alley, M., Hollingshead, M., Shoemaker, R. H., and Niederhuber, J. E. (2010). Complex display of putative tumor stem cell markers in the NCI60 tumor cell line panel. Stem Cells, 28, 649-660.
Sviderskaya, E. V., Hill, S. P., Evans-Whipp, T. J., Chin, L., Orlow, S. J., Easty, D. J., Cheong, S. C., Beach, D., DePinho, R. A., and Bennett, D. C. (2002). p16(Ink4a) in melanocyte senescence and differentiation. J Natl Cancer Inst, 94, 446-454.
Taliaferro-Smith, L., Nagalingam, A., Zhong, D., Zhou, W., Saxena, N. K., and Sharma, D. (2009). LKB1 is required for adiponectin-mediated modulation of AMPK-S6K axis and inhibition of migration and invasion of breast cancer cells. Oncogene, 28, 2621-2633.
Tanaka et al. (1994). J Gen Virol, 75, 2691-2698.
Telenius et al. (1992). Genomics, 13, 718-725.
Tijssen (1993). Laboratory Techniques in Biochemistry and Molecular Biology-Hybridization with Nucleic Acid Probes, Elsevier, New York, N.Y.
Tusher et al. (2001). Proc Natl Acad Sci USA, 98, 5116-5121.
U.S. Patent Application No. 2011/0119776.
U.S. Pat. No. 4,729,947.
U.S. Pat. No. 5,143,854.
U.S. Pat. No. 5,207,880.
U.S. Pat. No. 5,230,781.
U.S. Pat. No. 5,346,603.
U.S. Pat. No. 5,360,523.
U.S. Pat. No. 5,534,125.
U.S. Pat. No. 5,571,388.
U.S. Pat. No. 5,800,992.
U.S. Pat. No. 5,837,832.
U.S. Pat. No. 5,846,717.
U.S. Pat. No. 5,871,918.
U.S. Pat. No. 5,968,745.
U.S. Pat. No. 5,974,164.
U.S. Pat. No. 5,985,557.
U.S. Pat. No. 5,994,069.
U.S. Pat. No. 6,001,567.
U.S. Pat. No. 6,017,696.
U.S. Pat. No. 6,066,457.
U.S. Pat. No. 6,086,737.
U.S. Pat. No. 6,090,543.
U.S. Pat. No. 6,127,127.
U.S. Pat. No. 6,162,603.
U.S. Pat. No. 6,185,561.
U.S. Pat. No. 6,229,911.
U.S. Pat. No. 6,245,508.
Vankerckhoven et al. (1994). J Clin Microbiol, 30, 750-753.
Vignali (2000). J Immunol Methods 243, 243-255.
Wang et al. (1998). Proc Natl Acad Sci USA, 86, 9717-9721.
Williams (1989). BioTechniques, 7, 762-769.
Williams et al. (1990). Nuc Acids Res, 18, 6531-6535.
WO 1995/11755.
WO 1997/14028.
W01999/32660.
WO 1999/19515.
WO 1999/32660.
WO 2001/13120.
WO 2001/14589.
WO 2004/046098.
WO 2004/110244.
WO 2006/089268.
WO 2007/001324.
WO 2007/056332.
WO 2007/070252.
Worley et al. (2000) in Shena, ed., Microarray Biochip Technology, pp. 65-86, Eaton Publishing, Natick, Mass.
Zeng, P. Y., and Berger, S. L. (2006). LKB1 is recruited to the p21/VVAF1 promoter by p53 to mediate transcriptional activation. Cancer Res, 66, 10701-10708.
Zheng, B., Jeong, J. H., Asara, J. M., Yuan, Y. Y., Granter, S. R., Chin, L., and Cantley, L. C. (2009). Oncogenic B-RAF negatively regulates the tumor suppressor LKB1 to promote melanoma cell proliferation. Molecular cell, 33, 237-247.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of predicting a melanoma prognosis, the method comprising:
(a) detecting one or more of the following in a biological sample comprising melanoma cells obtained from a melanoma of a subject:
(i) the presence or absence of a LKB1 mutation, or a LKB1 expression level;
(ii) a YES expression level, a YES phosphorylation level, or both; and
(iii) a CD24 expression level; and
(b) predicting a melanoma prognosis based on the detecting of step (a).

2. A method of predicting a response to a therapy by a melanoma in a subject having the melanoma and receiving the therapy, the method comprising:
(a) detecting one or more of the following in a biological sample comprising melanoma cells obtained from a melanoma of a subject:
(i) the presence or absence of a LKB1 mutation, or a LKB1 expression level;
(ii) a YES expression level, a YES phosphorylation level, or both; and
(iii) a CD24 expression level; and
(b) predicting a response to the therapeutic based on the detecting of step (a).

3. A method for managing treatment of a subject with melanoma, the method comprising:
(a) detecting one or more of the following in a biological sample comprising melanoma cells obtained from a melanoma of a subject:
(i) the presence or absence of a LKB1 mutation, or a LKB1 expression level;
(ii) a YES expression level, a YES phosphorylation level, or both; and
(iii) a CD24 expression level; and
(b) managing treatment of the subject based on the detecting of step (a).

4. The method of any one of claims 1-3, wherein the presence of an LKB1 mutation or of a reduced level of expression of LKB1 is indicative of a negative prognosis, a resistance to the therapy, or suggests an altered treatment choice.

5. The method of any one of claims 1-3, wherein the absence of an LKB1 mutation or of a reduced level of expression of LKB1 is indicative of a positive prognosis, a lack of resistance to the therapy, or suggests an altered treatment choice.

6. The method of any one of claims 1-3, wherein an elevated level of YES expression, YES phosphorylation, or both, is indicative of a negative prognosis, a resistance to the therapy, or suggests an altered treatment choice.

7. The method of any one of claims 1-3, wherein the absence of an elevated level of YES expression, YES phosphorylation, or both, is indicative of a positive prognosis, a lack of resistance to the therapy, or suggests an altered treatment choice.

8. The method of any one of claims 1-3, wherein an elevated level of CD24 expression is indicative of a negative prognosis, a resistance to the therapy, or suggests an altered treatment choice.

9. The method of any one of claims 1-3, wherein the absence of an elevated level of CD24 expression is indicative of a positive prognosis, a lack of resistance to the therapy, or suggests an altered treatment choice.

10. The method of any one of claims 1-3, further comprising assessing a risk of an adverse outcome of a subject with melanoma.

11. The method of any one of claims 1-3, further comprising predicting a clinical outcome of a treatment in a subject diagnosed with melanoma.

12. The method of any one of claims 1-3, wherein an expression level is determined by a PCR-based method, a microarray based method, or an antibody-based method.

13. The method of any one of claims 1-3, wherein an expression level is normalized relative to an expression level of one or more reference genes.

14. The method of any one of claims 1-3, comprising comparing the expression level to a standard.

15. The method of claim 2 or claim 3, where the therapy or treatment is selected from the group consisting of surgical resection of the melanoma, chemotherapy, molecular targeted therapy, immunotherapy, and combinations thereof.

16. A method of treating melanoma in a subject in need thereof, comprising administering to the subject an effective amount of an inhibitor of a SRC family kinase, optionally a targeted inhibitor of a SRC family kinase, optionally YES, to treat a melanoma in the subject.

17. The method of any one of claims 1-3 and 16, wherein the subject is a mammal.

18. An array comprising polynucleotides hybridizing to at least two genes selected from the group consisting of LKB1, YES, and CD24 or comprising specific peptide or polypeptide gene products of at least two of LKB1, YES, and CD24.

19. A kit comprising one or more binding molecules for a gene selected from the group consisting of LKB1, YES, and CD24 and/or for a peptide or polypeptide gene product of LKB1, YES, or CD24.

20. A method of selecting a therapy for a melanoma in a subject in need of treatment for the melanoma, comprising providing a subject suffering from a melanoma wherein LKB1, YES and/or CD24 status for the subject's melanoma has been assessed; and selecting a therapy for the subject based on the status of LKB1, YES and/or CD24.

21. The method of claim 20, comprising administering to the subject an effective amount of a therapeutic agent to treat the melanoma in the subject based on the status of LKB1, YES and/or CD24.

22. A method of treating melanoma in a subject in need thereof, comprising providing a subject suffering from a melanoma wherein LKB1, YES and/or CD24 status for the subject's melanoma has been assessed; and administering to the subject an effective amount of a therapeutic agent to treat the melanoma in the subject based on the LKB1, YES and/or CD24 status.

23. The method of claim 20 or 22, wherein the subject is a mammal.
